(19) United States
(12) Patent Application Publication
(10) Pub. No.: US 2004/0186566 A1
(43) Pub. Date: Sep. 23, 2004

(54) Body tissue remodeling methods and apparatus

(76) Inventors: Paul J. Hindrichs, Plymouth, MN (US); Steven D. Kruse, St. Michael, MN (US); Todd A. Krinke, Rockford, MN (US); Michael P. Brenzel, St. Paul, MN (US); Matthew M. Quest, Woodbury, MN (US); Kenton J. Zehr, Rochester, MN (US); Todd A. Berg, Stillwater, MN (US); John Logan, Plymouth, MN (US); Stephen T. Kuehn, Woodbury, MN (US)

Correspondence Address: FISH & NEAVE, 1251 AVENUE OF THE AMERICAS, 50TH FLOOR, NEW YORK, NY, 10020-1105, US

(21) Appl. No.: 10/803,287

(22) Filed: Mar. 17, 2004

Related U.S. Application Data
(60) Provisional application No. 60/455,810, filed on Mar. 18, 2003.
(60) Provisional application No. 60/519,011, filed on Nov. 10, 2003.

(51) Int. Cl. A61F 2/24; A61B 17/068

(52) U.S. Cl. 623/2.37; 623/1.36; 600/37; 227/175.1

(57) ABSTRACT

A patient's soft body tissue can be remodelled by implanting first and second anchor structures in the tissue at respective first and second spaced locations. A linking structure between the anchor structures is then operated to change the distance between the first and second anchor structures. Examples of use are repair of a patient's mitral and/or tricuspid valve(s) and/or remodeling of a patient's left ventricle.

This application claims the benefit of U.S. provisional patent applications 60/455,810, filed Mar. 18, 2003, and 60/519,011, filed Nov. 10, 2003, both of which are hereby incorporated by reference herein in their entireties.

### BACKGROUND OF THE INVENTION

This invention relates to body tissue remodeling, e.g., to changing the size and/or shape of one or more body tissue structures of a patient. Although most of the examples discussed in detail herein relate to remodeling cardiac tissue structures, it will be understood that the invention is also applicable to remodeling other relatively soft (i.e., non-bone) tissue structures of a patient.

Various conditions can cause portions of the heart to enlarge undesirably. For example, the left ventricle can become distended or the annulus of tissue that is at the base of the mitral valve (between the left atrium and the left ventricle) can enlarge annularly. Either of these conditions can prevent the leaflets of the mitral valve from closing or sealing properly, which adversely affects the blood-pumping capability of the heart, e.g., by allowing blood to regurgitate from the left ventricle into the left atrium. Enlargement of the left ventricle may also have other adverse consequences for the patient. Remodeling may be one way to reverse the effects of an undesirable tissue structure enlargement and thereby restore proper functioning to that structure. Even without enlargement of a tissue structure, the structure may lose its ability to function properly. For example, a mitral valve may leak for any of a number of reasons that are not due to enlargement of any part of the heart. Nevertheless, remodeling (e.g., size reduction or shape change) may be effective in stopping such leakage. Another example is remodeling cardiac tissue structures adjacent the tricuspid valve (between the right atrium and the right ventricle) to improve performance of that valve.

Various surgical techniques are known for repairing areas of the heart such as those mentioned above. However, this is relatively major surgery, and it would be desirable to have less invasive techniques for treating these kinds of conditions. Example of less invasive techniques are those that are percutaneous, thorascopic, laparoscopic, endoscopic, or the like. But even if surgery is still required, methods and apparatus that make such surgery faster and/or easier, and/or that produce better, more predictable, and more reliable results are still desired.

It is therefore an object of this invention to provide new and improved techniques (including new and improved methods and/or apparatus) for providing tissue remodeling.

### SUMMARY OF THE INVENTION

This and other objects of the invention are accomplished in accordance with the principles of the invention by providing methods and/or apparatus for implanting at least two anchor structures in a patient's body tissue, and then drawing those two anchor structures (and the adjacent tissue) toward one another using a linking or cinching structure that extends between the anchor structures. This shortens the distance between the anchor structures and the adjacent tissue and thereby provides desired tissue remodeling.

Illustrative examples of the invention include methods and/or apparatus for percutaneously implanting at least two anchor structures in the heart adjacent the mitral valve annulus. At least one of the anchor structures is supplied to its final location via at least a portion of the coronary sinus. The final location of that anchor structure may be the coronary sinus, a diagonal branching off the coronary sinus, or the great cardiac vein. The other anchor structure may also be supplied to its final location as described above, or its final location may be in the right atrium, e.g., near the ostium of the coronary sinus. The two anchor structures are secured to heart tissue at their final locations. The two anchor structures are interconnected by a linking or cinching structure, the length of which can be changed and then fixed. Preferably, after the anchor structures have been secured in their final locations, the length of the linking structure between the anchor structures can be shortened and then fixed. This reduces the distance between the two anchor structures, thereby shortening the portion of the mitral valve annulus that is adjacent to and between the anchor structures. Shortening any portion (or portions) of the mitral valve annulus in this way reduces the overall circumference of that annulus. As suggested above, this technique may be applied adjacent more than one portion of the mitral valve annulus. The invention may also be applied to other parts of the heart (e.g., in the left ventricle or adjacent the tricuspid valve), to other body tissue structures, and by approaches that are other than percutaneous (e.g., thorascopically, surgically, etc.).

The invention also includes various constructions of anchor structures and apparatus for percutaneously or otherwise delivering and implanting those anchor structures. The invention also includes various constructions of linking structures between anchor structures, and various structures for changing and/or fixing the length of such linking structures, including percutaneously or otherwise delivering and operating those structures. And the invention includes instrumentation for delivering and implanting anchor structures, and for operating linking or cinching structures between anchor structures. This instrumentation may be adapted for use percutaneously, thorascopically, laparoscopically, surgically, or in any other desired manner.

Further features of the invention, its nature and various advantages, will be more apparent from the accompanying drawings and the following detailed description of the preferred embodiments.

### BRIEF DESCRIPTION OF THE DRAWINGS

FIG. 1 is a simplified, substantially horizontal, cross sectional view of a patient's heart showing illustrative treatment in accordance with the invention. (Another way to describe this cross section is as substantially parallel to the mitral valve annulus.)

FIG. 2 is another view similar to FIG. 1 showing an early stage in an illustrative treatment in accordance with the invention. (All of the FIGS. herein show the patient's heart having the same basic configuration, both before and after treatment in accordance with the invention. It will be understood, however, that aspects of the patient's heart (e.g., the mitral valve annulus) may be enlarged prior to treatment and changed in size and/or shape as a result of treatment in accordance with the invention.)

FIG. 3 is still another view similar to FIG. 1 showing a later stage in an illustrative treatment in accordance with the invention.

FIG. 4 is a simplified, substantially vertical, cross sectional view of a portion of the heart shown in FIG. 3.

FIG. 5 is a simplified perspective view of an illustrative embodiment of a component constructed in accordance with the invention.

FIG. 6 is yet another view similar to FIG. 1 showing a still later stage in an illustrative treatment in accordance with the invention.

FIG. 7 is another simplified, substantially vertical, cross sectional view of the heart shown in FIG. 6 showing a still later stage in an illustrative treatment in accordance with the invention.

FIG. 8 is still another view similar to FIG. 1 showing a yet later stage in an illustrative treatment in accordance with the invention.

FIG. 9 is a simplified vertical section of a portion of a heart showing a condition resulting from treatment that can often be avoided in accordance with the invention.

FIG. 10 is yet another view similar to FIG. 1 showing a still later stage in an illustrative treatment in accordance with the invention.

FIG. 11 is another view similar to FIG. 1 showing an alternative treatment in accordance with the invention.

FIG. 12 is yet another view similar to FIG. 1 showing another alternative treatment in accordance with the invention.

FIG. 13 is still another view similar to FIG. 1 showing still another alternative treatment in accordance with the invention.

FIG. 14 is a simplified perspective view showing another illustrative embodiment of apparatus in accordance with the invention.

FIG. 15 is a view similar to a portion of FIG. 14 showing yet another illustrative embodiment of apparatus in accordance with the invention.

FIG. 16 is another view similar to FIG. 14 showing still another illustrative embodiment of apparatus in accordance with the invention.

FIG. 17 is still another view similar to a portion of FIG. 14 showing yet another illustrative embodiment of apparatus in accordance with the invention.

FIGS. 18 and 19 are more views similar to portions of FIG. 14 showing still another illustrative embodiment of apparatus in accordance with the invention.

FIG. 19a is a simplified view, partly in section, of illustrative apparatus for installing an anchor structure of the general type illustrated by FIGS. 18 and 19.

FIG. 20 is a simplified perspective view showing an illustrative embodiment of a possible apparatus component in accordance with the invention.

FIG. 21 is another view generally similar to FIG. 20 showing another illustrative embodiment of a possible apparatus component in accordance with the invention.

FIG. 22 is another view similar to FIG. 14, but showing a different operating condition of the apparatus.

FIG. 23 is another view similar to FIG. 22 showing a subsequent operating condition of the apparatus.

FIG. 24 is still another view similar to FIG. 23 showing a still later operating condition of the apparatus.

FIG. 25 is yet another view similar to FIG. 24 showing an even later operating condition of the apparatus.

FIG. 26 is a simplified elevational view of apparatus like that shown in FIG. 14.

FIG. 27 is another simplified elevational view of what is shown in FIG. 26.

FIG. 28 is a simplified schematic view of a portion of a patient's anatomy that is useful in explaining certain aspects of the invention.

FIG. 29 is a view similar to FIG. 5 showing another illustrative embodiment of apparatus in accordance with the invention.

FIG. 30 is a simplified sectional view showing another illustrative embodiment of apparatus in accordance with the invention.

FIG. 31 is another view similar to FIG. 30 showing further illustrative apparatus in accordance with the invention.

FIG. 32 is another view similar to FIG. 31 showing a later stage in use of the illustrative apparatus in accordance with the invention.

FIG. 33 is another view similar to FIG. 32 showing a still later stage in use of the illustrative apparatus in accordance with the invention.

FIG. 34 is another view similar to FIG. 33 showing an even later stage in use of the illustrative apparatus in accordance with the invention.

FIG. 35 is s simplified elevational view of another illustrative embodiment of apparatus in accordance with the invention.

FIG. 36 is another view of what is shown in FIG. 35 taken along the line 36-36 in FIG. 35.

FIG. 37 is a view similar to FIG. 36 showing a later stage in use of the embodiment shown in FIGS. 35 and 36.

FIG. 38 is a view similar to FIG. 4 showing another example of use of the invention.

FIG. 39 is another view similar to FIG. 38 showing a later stage in the FIG. 38 example.

FIG. 40 is another view similar to FIG. 39 showing another illustrative embodiment of a procedure like that shown in FIG. 39 in accordance with the invention.

FIG. 41 is another view similar to FIG. 1 showing another illustrative use of the invention.

FIG. 42 is another view similar to FIG. 1 showing still another illustrative use of the invention.

FIG. 43 is a more detailed, but still simplified, view of a portion of what is shown in FIG. 4.

FIG. 44 is a simplified elevational view of a portion of illustrative apparatus in accordance with the invention.

FIG. 45 is another simplified elevational view taken along the line 45-45 in FIG. 44.

FIG. 46 is another view similar to FIG. 44 showing another illustrative embodiment of apparatus of the general type shown in FIG. 44.

### DETAILED DESCRIPTION

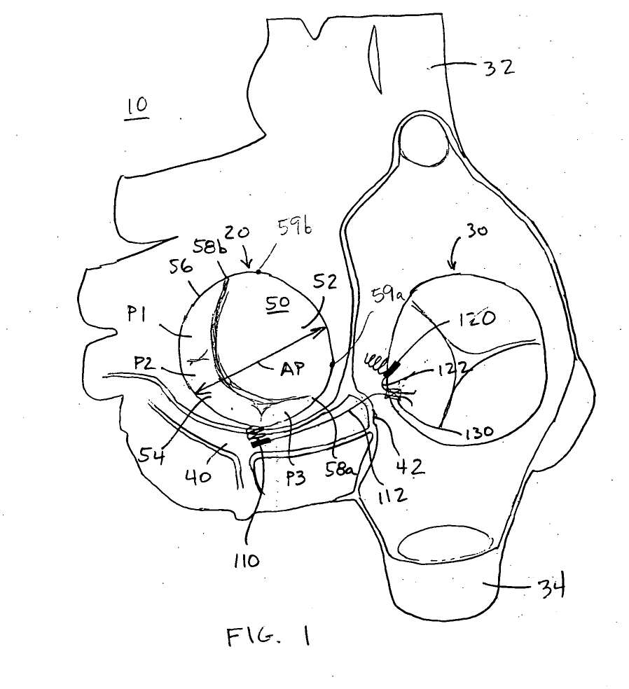

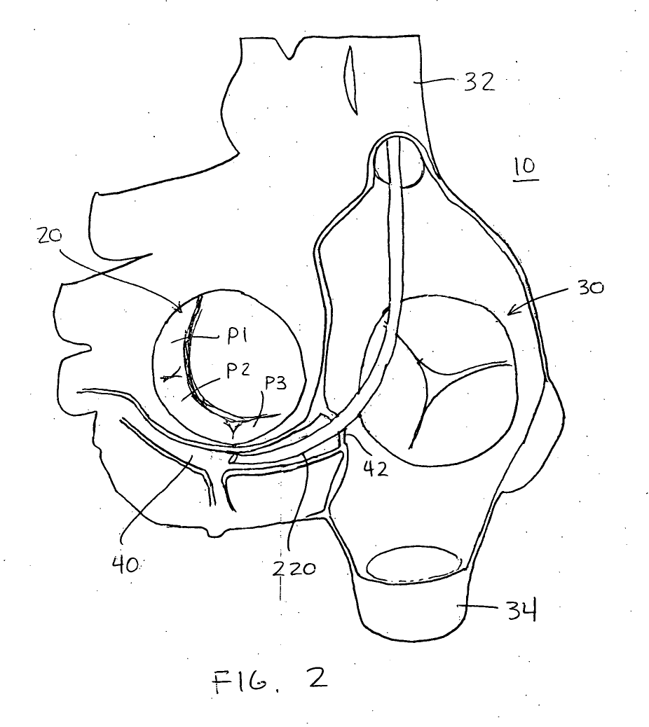

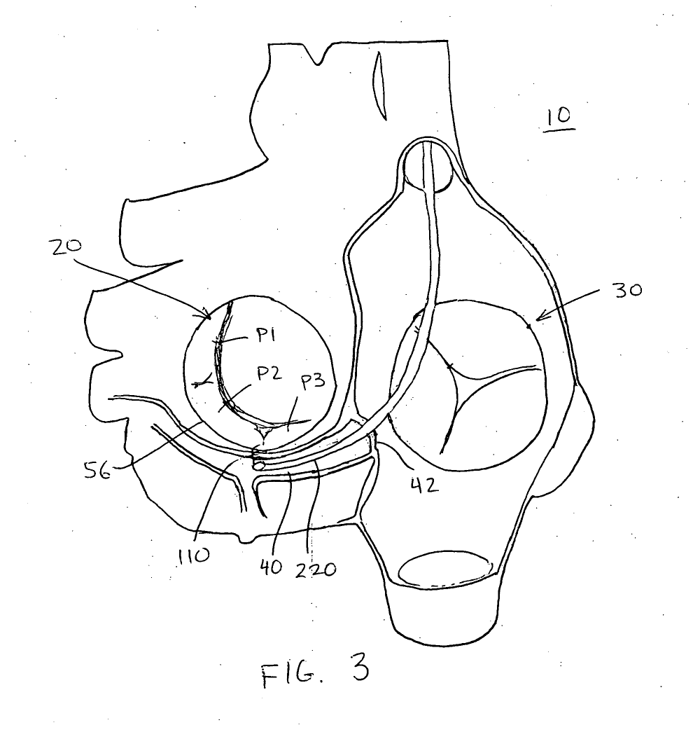

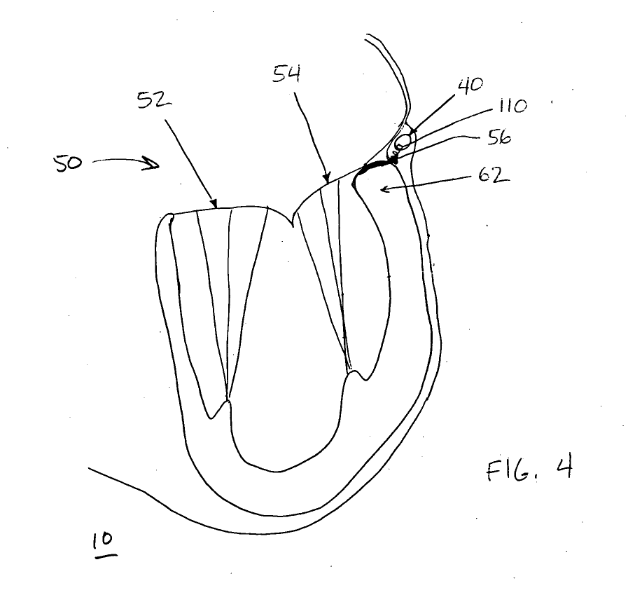

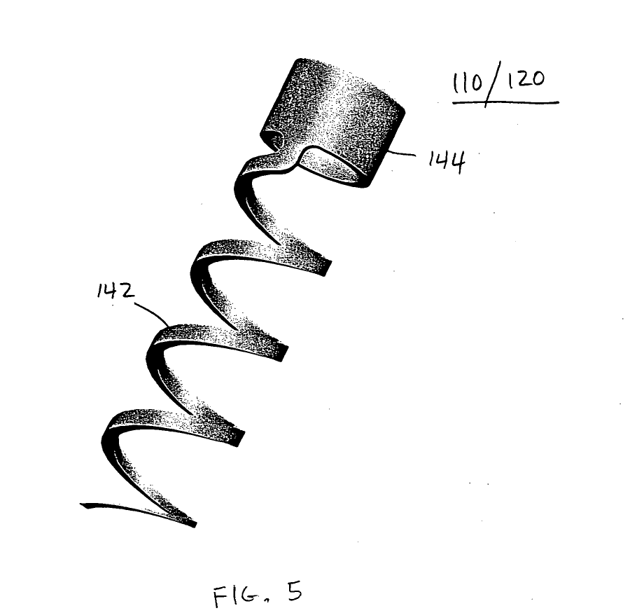

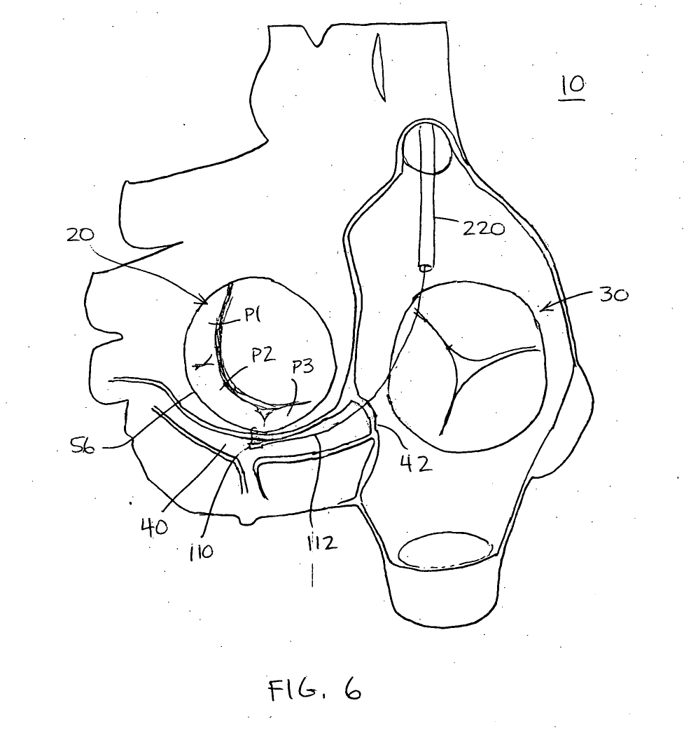

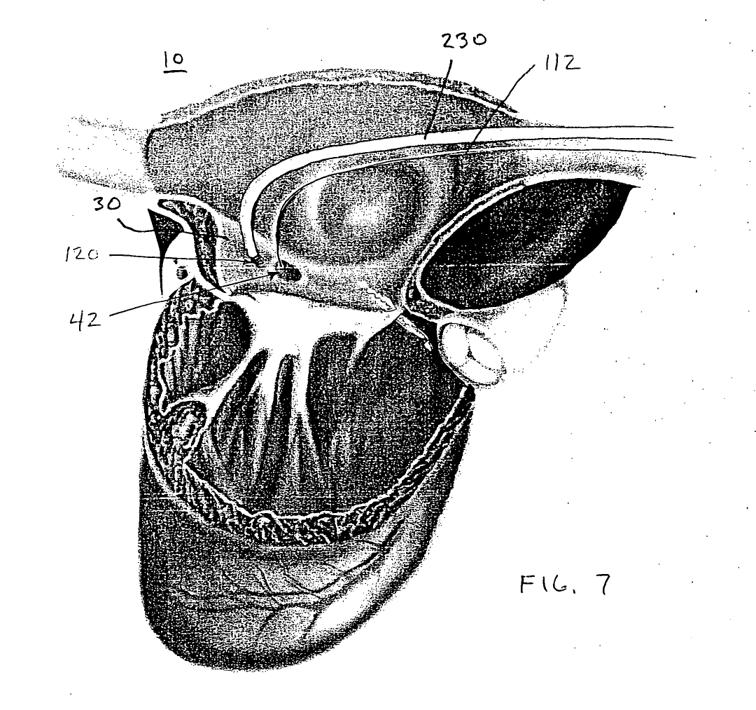

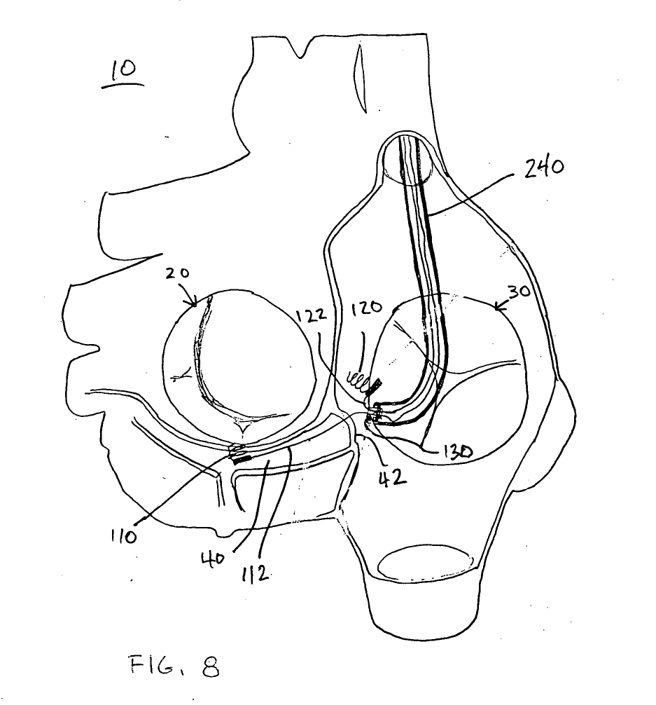

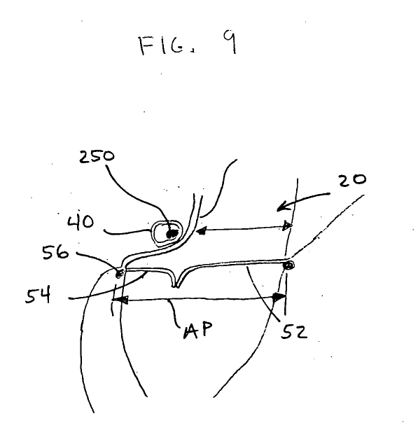

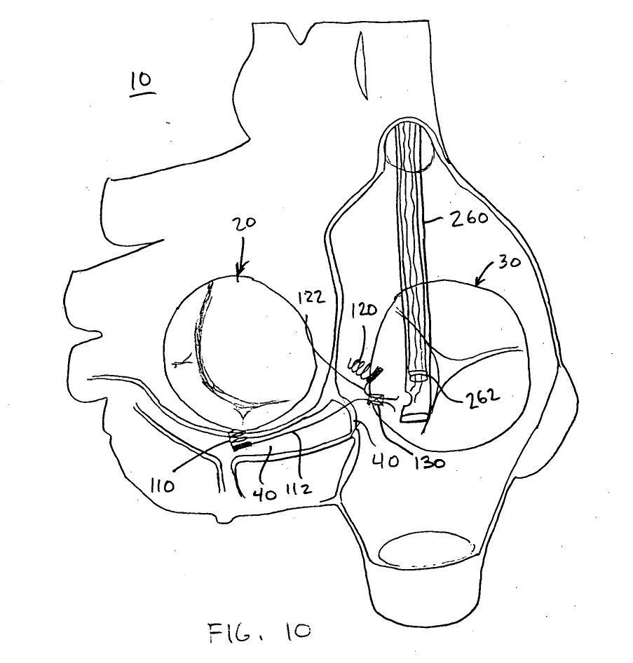

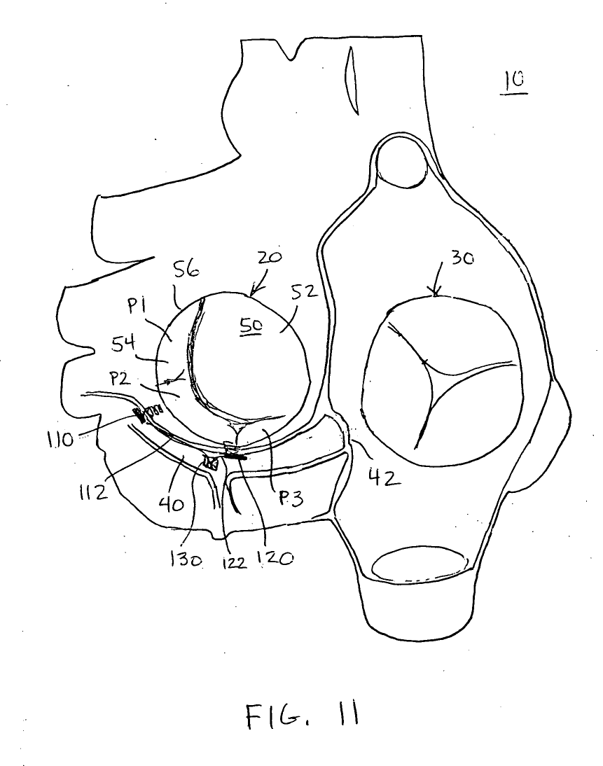

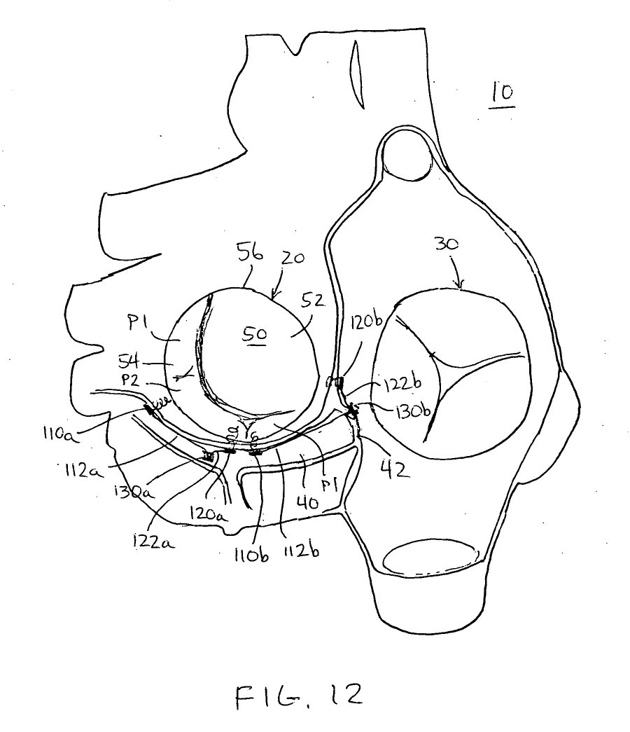

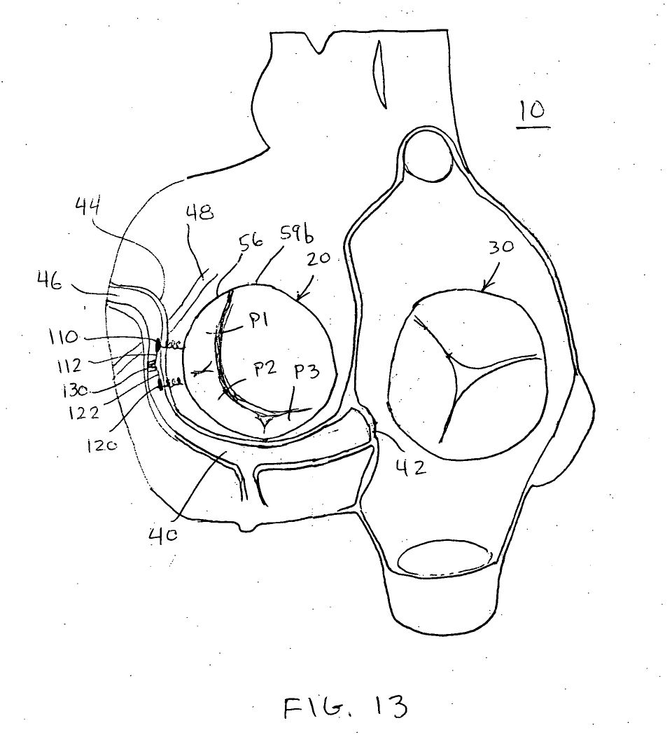

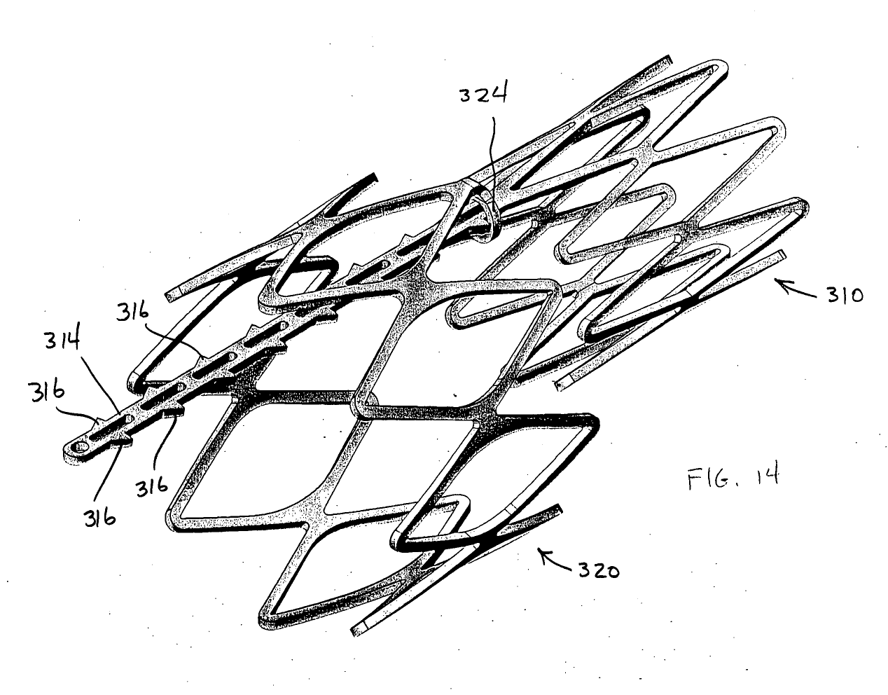

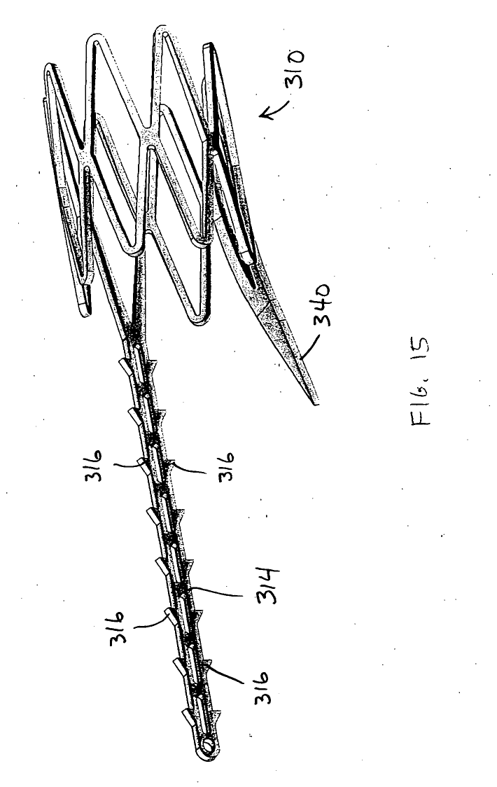

FIG. 1 shows a heart 10 that has been treated in accordance with a first illustrative embodiment of the invention. Among the heart structures shown in FIG. 1 are left atrium 20, right atrium 30, and coronary sinus 40. The ostium 42 of coronary sinus 40 communicates with the interior of right atrium 30. At the bottom of left atrium 20 is mitral valve 50. Mitral valve 50 includes anterior leaflet 52 and posterior leaflet 54 (having three segments P1, P2, and P3). Posterior leaflet segment P3 is closest to the ostium 42 of coronary sinus 40. Posterior leaflet segment P2 is farther from ostium 42 as one progresses along coronary sinus 40. And posterior leaflet segment Pi is still farther from ostium 42 around the annulus 56 of mitral valve 50. The bases of leaflets 52 and 54 (or segments P1-P3) are joined to mitral valve annulus 56. The bases of leaflets 52 and 54 are immediately adjacent to one another at commissure 58a (near the ostium 42 of coronary sinus 40) and at opposite commissure 58b (remote from ostium 42). Coronary sinus 40 extends from ostium 42 a significant way around the outside of the posterior of left atrium 20 at a level near the level of mitral valve annulus 56. (Mitral valve annulus 56 actually tends to be somewhat saddle shaped and therefore not in any one geometric plane. For simplicity in the present discussion, however, it will generally be assumed that mitral valve annulus 56 has an average level that can be thought of as approximately planar. References to other structures being “above” or “below” the mitral valve annulus or the level of the mitral valve annulus or the like will be understood to refer to above or below this approximately planar, average level of the annulus.)

A normal mitral valve 50 opens selectively to allow blood to flow from left atrium 20 down into the left ventricle (not visible in FIG. 1). A normal mitral valve 50 closes or seals to at least substantially prevent blood from flowing back up into left atrium 20 when the left ventricle contracts to force blood out into the aorta. If mitral valve 50 is not closing or sealing properly, it can allow blood to regurgitate from the contracting left ventricle back into left atrium 20, which is or which can lead to a serious heart problem for the patient. Mitral valve 50 may not close or seal properly for any of several reasons, which may be described variously by various people skilled in the art. For example, mitral valve 50 may not close because of some enlargement. Various people skilled in the art may characterize this as an enlargement of mitral valve annulus 56, or as an enlargement of the area of valve 50 bounded by annulus 56, or as an enlargement of the anterior-posterior dimension AP of valve 50. Remodeling of the heart in the vicinity of mitral valve annulus 56 in accordance with this invention can be effective in causing mitral valve 50 to again close or seal properly. Even without enlargement of any portion of the heart, mitral valve 50 may not be closing or sealing properly due to any of several other undesirable conditions, and remodeling in the vicinity of annulus 56 in accordance with this invention may cause to valve 50 to close or seal properly.

As has been mentioned, FIG. 1 also shows a first illustrative embodiment of treatment of the heart in accordance with the invention. This illustrative treatment embodiment includes implanting a first anchor structure 110 in coronary sinus 40, in this case near the junction between leaflet segments P2 and P3 (“the P2/P3 junction”). (Although anchor structure 110 is here preliminarily described as being implanted in coronary sinus 40, it will be understood that anchor structure 110 preferably extends through the wall of the coronary sinus into other adjacent tissue that helps to hold structure 110 securely in place in the heart. Details regarding this are provided later in this specification.) The illustrative treatment being described further includes implanting a second anchor structure 120 in the wall of right atrium 30, e.g., near the ostium 42 of coronary sinus 40. (Again, more details regarding preferred placement of and tissue engagement by anchor structure 120 will be provided later in this specification.)

Anchor structure 110 includes a flexible member 112 that extends from it toward anchor structure 120. Anchor structure 120 similarly includes a flexible member 122 that extends from it toward anchor structure 110. A cinching structure 130 engages both of flexible members 112 and 122 between anchor structures 110 and 120 in such a way that the cinching structure cooperates with the flexible members to prevent anchor structures 110 and 120 from moving farther apart than is shown in FIG. 1. (As will be illustrated more fully later in this specification, the linking structure between anchors 110 and 120, which in this embodiment includes two flexible members 112 and 122 and cinching structure 130, can be constructed in many other ways. For example, in other embodiments described later a cinchable linking structure between anchor structures is provided by a relatively inflexible, elongated, ratchet-type member extending from one anchor structure through an eyelet on the other anchor structure.)

As will be described in more detail below, anchor structures 110 and 120 are implanted, and then they are typically pulled toward one another using flexible members 112 and 122. (Actually, in the embodiment shown in FIG. 1, pulling on flexible members 112 and 122 through cinching structure 130 tends to pull anchor structure 110 and its adjacent tissue toward anchor structure 120 and its adjacent tissue, because in this embodiment anchor structure 120 is secured to a relatively stiff part of the heart, while anchor structure 110 is secured to a relatively movable part of the heart. This will also be explained in more detail below.) Shortening the distance between anchor structures 110 and 120 shortens the segment of mitral valve annulus 56 between them. This shortens annulus 56 as a whole, thereby reducing the mitral valve area bounded by annulus 56 and also reducing dimension AP, as well as the commissure to commissure dimension (i.e., the distance between commissures 58a and 58b). When the desired spacing of anchor structures 110 and 120 has been achieved, cinching structure 130 holds that spacing of the anchor structures. The entirety of implanting elements 110, 112, 120, 122, and 130 and operating those elements to shorten mitral valve annulus 56 is preferably performed percutaneously. The preferred percutaneous approach is via catheter-type instrumentation introduced into the patient's heart via blood vessels (veins or arteries) leading to the heart. (Although percutaneous is the preferred technique, any of the other techniques mentioned earlier in this specification can be used instead if desired.)

It will be noted in connection with FIG. 1 that there is a relatively straight line between anchor structures 110 and 120 (i.e., through the proximal portion of coronary sinus 40, ostium 42, and out into right atrium 30). (Throughout this specification terms like “proximal” and “distal” are used with reference to being closer to or farther from the physician or other person performing the procedure in a manner that is assumed to be percutaneous. However, these terms are only used for convenience and in a relative sense. It is not intended for use of these terms to be limiting in any way.) Shortening such a relatively straight segment of annulus 56 with the apparatus of this invention (which in this embodiment is also relatively straight between anchor structures 110 and 120) is advantageous because it is very efficient in reducing the overall length of annulus 56 and because it helps to avoid introducing other possibly less desirable distortions into the annulus and/or adjacent tissue structures (see, for example, FIG. 9, which is discussed later in this specification).

Another advantage of embodiments of the type illustrated by FIG. 1 is the following. Coronary sinus 40 tends to be slightly above mitral valve annulus 56. The point of attachment of flexible member 112 to anchor structure 110 is therefore typically above annulus 56. (It may be possible to lower anchor structure 110 by implanting it in ma downwardly extending tributary to coronary sinus 40. References herein to the coronary sinus will be understood to also include tributaries to the coronary sinus.) Anchor structure 120, on the other hand, can be advantageously implanted in the wall of right atrium 30 so that the point of attachment of flexible member 122 to anchor structure 120 is below coronary sinus ostium 42 and also below the level of mitral valve annulus 56. This means that the linking structure (elements 112, 122, and 130) between anchor structures 110 and 120 crosses the level of mitral valve annulus 56. The annulus-shortening effect of the apparatus is therefore neither wholly above nor wholly below annulus 56, which could produce some occlusion of the blood flow path to or from mitral valve 50, while less efficiently shortening annulus 56 as is desired from the apparatus. Instead, the net effect in accordance with this embodiment of the invention is approximately at the level of annulus 56. This net effect most efficiently applies the shortening at the annular plane. In this context “efficient” means that for a given amount of shortening of the apparatus, a maximum or nearly maximum amount of shortening of annulus 56 results.

Still another advantage of embodiments of the type illustrated by FIG. 1 is the following. Implanting the proximal anchor structure 120 in the right atrium secures that anchor structure to a relatively rigid part of the heart. By way of contrast, distal anchor structure 110, implanted in coronary sinus 40, is in a more compliant part of the heart. This means that when flexible members 112 and 122 are pulled through cinching structure 130, the tissue adjacent anchor structure 110 is pulled more toward anchor structure 120 than vice versa. The location of anchor structure 120 remains relatively fixed, while anchor structure 110 is drawn toward that location. This causes this embodiment of the invention to have an efficient effect on reducing the area of not only the P3 segment of the valve, but also in reducing the area of the P2 and P1 segments as well. The entire posterior portion of valve annulus 56 is pulled toward relatively fixed anchor structure 120, thereby reducing the area of all three posterior leaflet segments P1-P3, even though only segment P3 is immediately adjacent the apparatus.

It should also be mentioned that the parts of the apparatus that are left in coronary sinus 40 preferably do not block the coronary sinus, but instead leave that lumen open for continued return flow of blood to right atrium 30.

Although the shortening of mitral valve annulus 56 occurs primarily across posterior leaflet segment P3, both P1 and P2 leaflet areas are also advantageously reduced. This is so, for example, because the perimeters of all three segments P1-P3 are drawn toward structure 120 in the right atrium as mentioned earlier. There is also little or no risk of occlusion of or impingement on the circumflex artery, which typically crosses over or under coronary sinus 40 well distal of the P2/P3 junction.

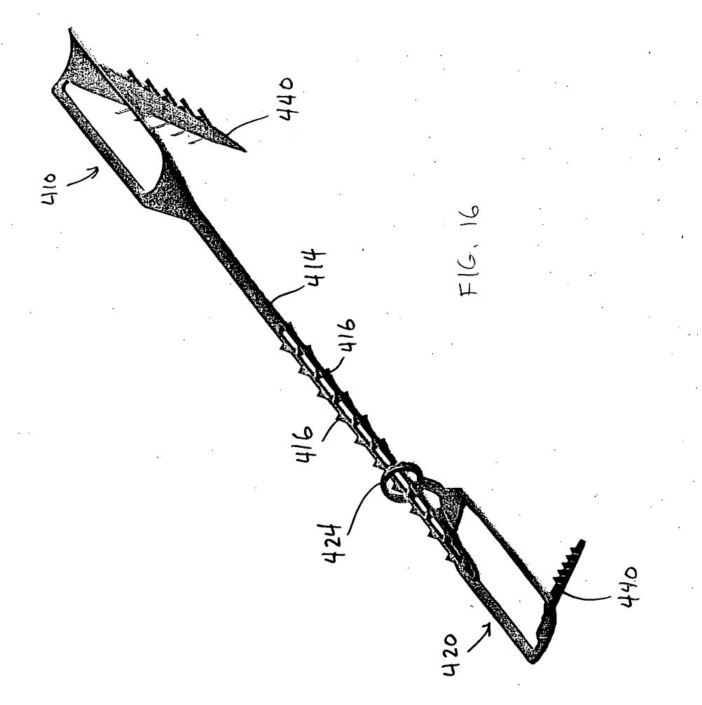

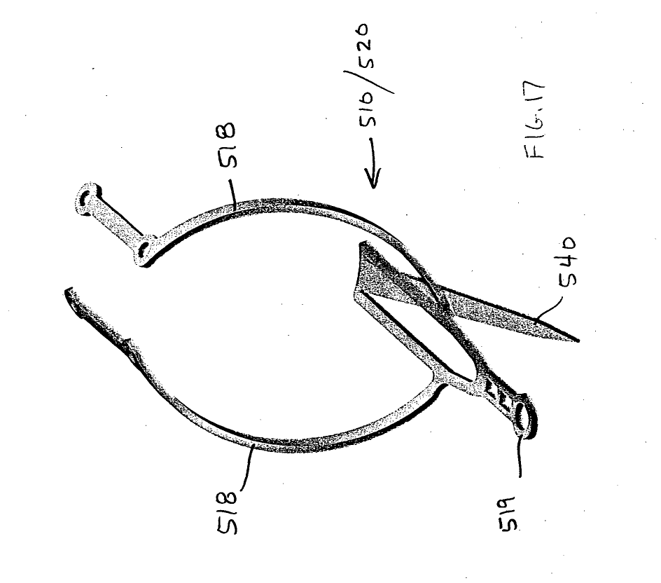

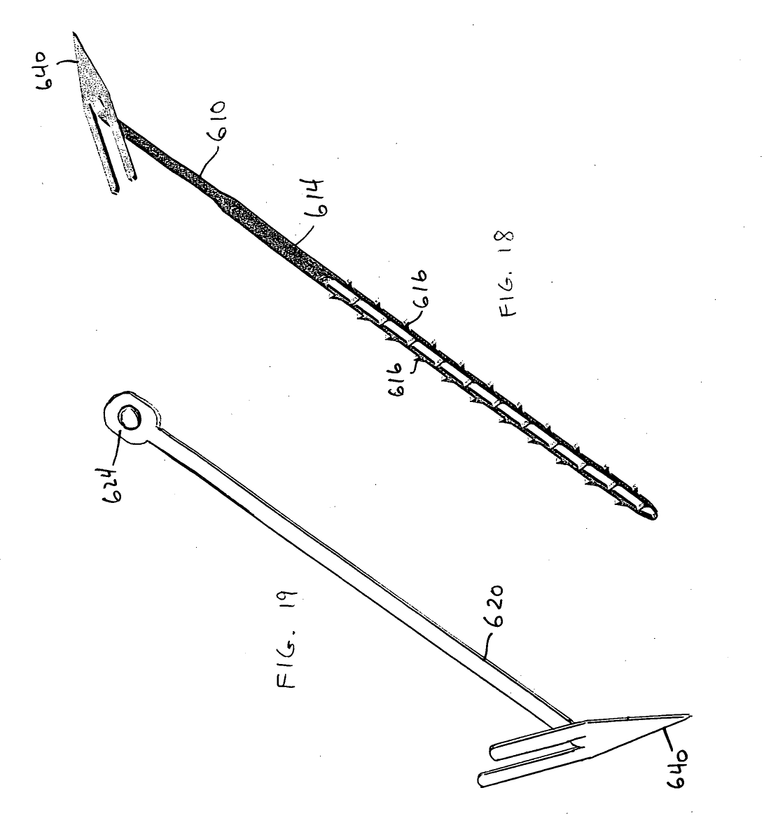

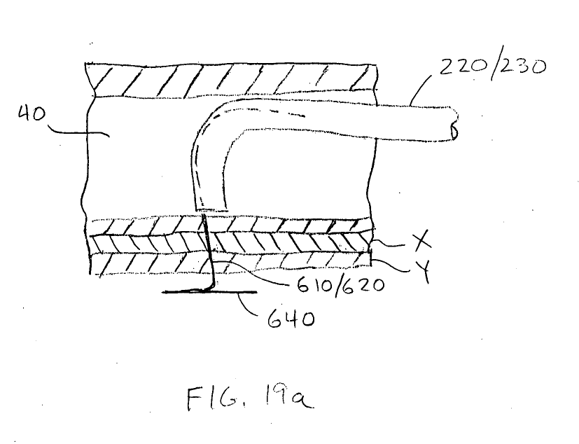

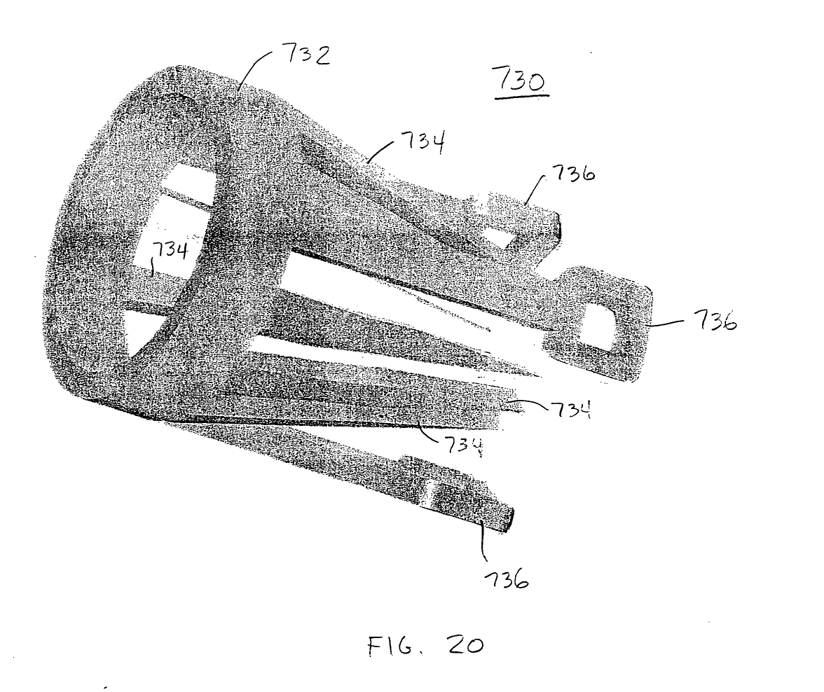

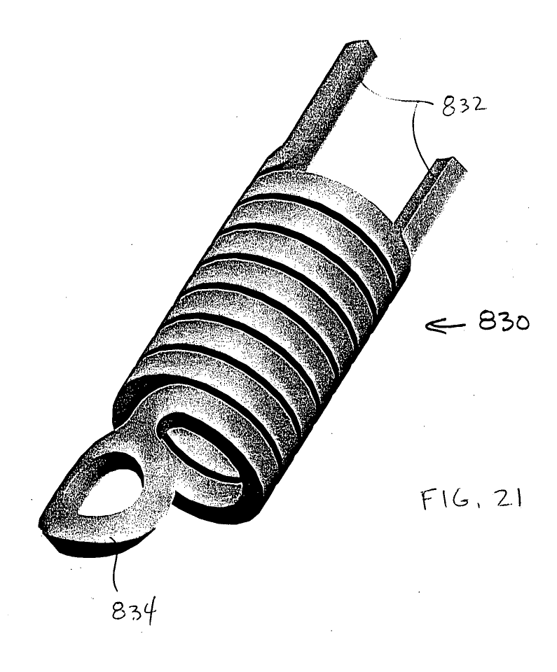

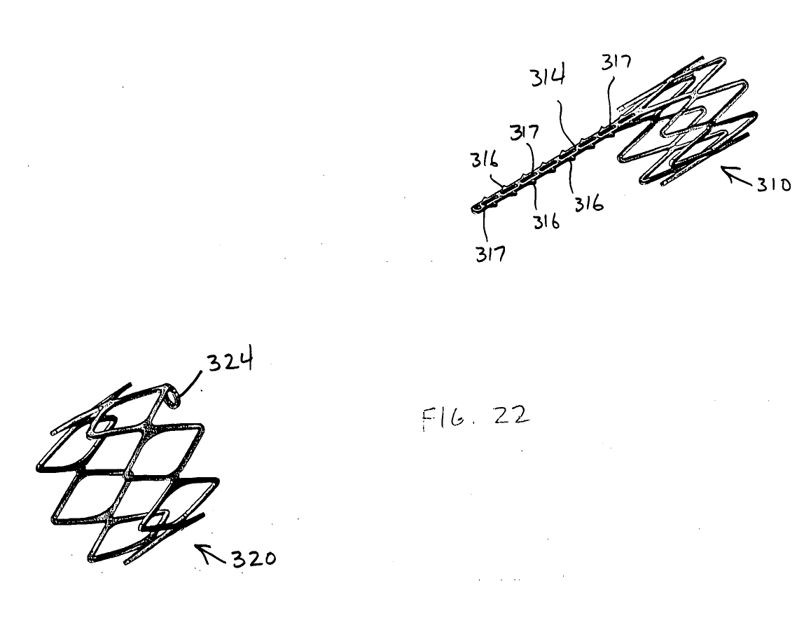

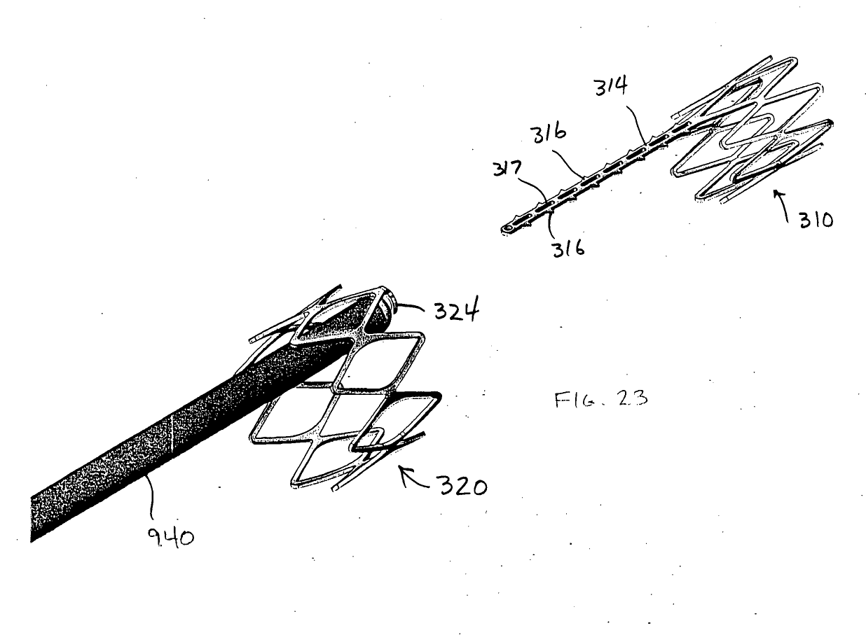

Illustrative methods and instrumentation for percutaneously implanting and operating mitral valve repair apparatus of the type shown in FIG. 1 is shown in FIG. 2 et seq. FIG. 2 shows an early stage in the procedure. In FIG. 2 catheter 220 has been introduced into right atrium 30 via superior vena cava 32. (A possible alternative approach is via inferior vena cava 34.) From right atrium 30, catheter 220 has been extended into the ostium 42 of coronary sinus 40 and along the coronary sinus to a location adjacent the P2/P3 junction. If desired, catheter 220 may follow a guide wire that has been previously introduced into coronary sinus 40 and perhaps lodged distally in the great cardiac vein.

The next aspect of the illustrative procedure being described is shown in FIG. 3. This is deployment of anchor structure 110 from a distal portion of catheter 220. Anchor structure 110 pierces through the wall of coronary sinus 40 and anchors into mitral annulus 56, ventricular myocardium 62 (FIG. 4), through the atrial wall into left atrium 20, into the pericardial space, or some combination of these structures. In the embodiment being described, anchor structure 110 is a helical screw (see also FIG. 5). The coil 142 of the screw is sized to penetrate down into the tissue of annulus 56 or the other structures mentioned above, and the head 144 of the screw sits in the bottom of coronary sinus 40. Alternatively, the head of the screw could be flush with or buried under the tissue surface. The screw embodiment shown in FIG. 5 is only one possibility for anchor structures 110/120 and there are many alternatives, several examples of which will be discussed in more detail later in this specification.

The next aspect of the illustrative procedure being discussed is shown in FIG. 6. This is proximal withdrawal of catheter 220 from coronary sinus 40 and ultimately out of the patient. Flexible member 112 (e.g., a band of fabric, polymer, or metal) extends from the head 144 (FIG. 5) of anchor structure 110 all the way out of the patient. Band 112 can be of similar composition and size as annuloplasty rings currently used to repair regurgitant mitral valves. Band 112 can change in size, shape, and/or composition along its length. The relatively small head 144 of screw 110 may protrude into coronary sinus 40. Alternatively, head 144 could be buried flush with the wall of the coronary sinus or completely embedded into the tissue below the coronary sinus as was mentioned earlier.

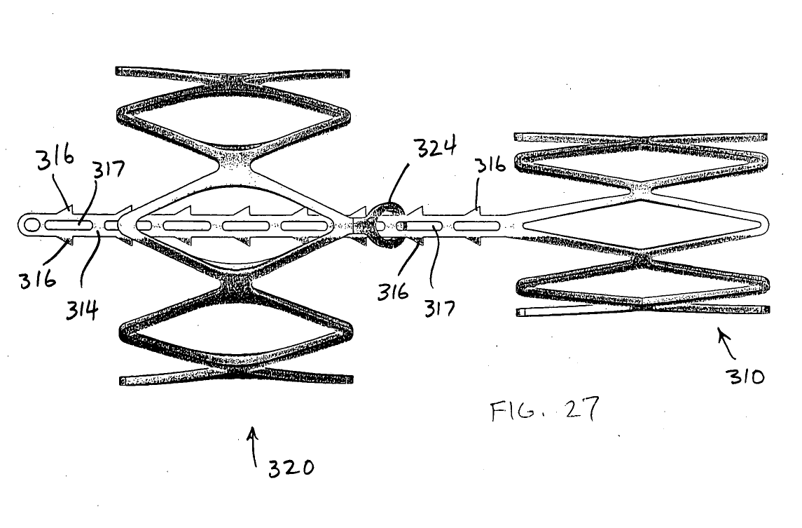

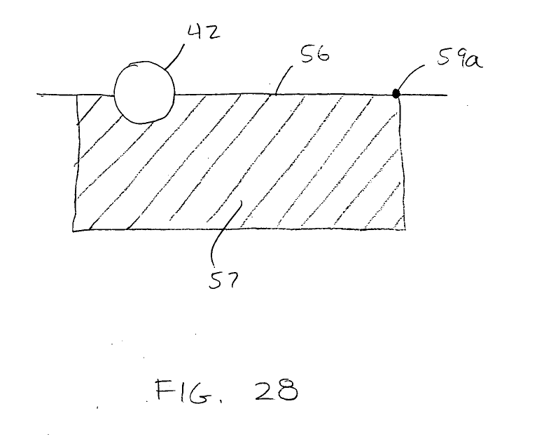

The next aspect of the illustrative procedure being described is shown in FIG. 7. A still further catheter 230 containing proximal anchor structure 120 is advanced into contact with the wall of right atrium 30 at or below the level of the level of mitral annulus 56 (e.g., FIG. 1). Proximal anchor 120 is deployed from catheter 230 into the right atrial wall below coronary sinus ostium 42, and preferably at or below the level of mitral annulus 56 in the region between trigone 59a (FIG. 1) and coronary sinus ostium 42, or directly through the right atrial wall into trigone 59a. Another possibility would be to locate anchor structure 120 just inside the coronary sinus ostium. FIG. 28 is a simplified schematic diagram showing a range 57 of preferred locations for anchor structure 120 in right atrium 30. Range 57 is basically below the level of annulus 56 and between coronary sinus ostium 42 and trigone 59a, although it may also extend a short distance to the side of ostium 42 remote from trigone 59a as shown in FIG. 28. Again, in sinus 40 just inside ostium 42 is also a possibility. Proximal anchor structure 120 preferably anchors into mitral annulus 56, ventricular myocardium 62 (FIG. 4), the atrial myocardium, or a combination of these tissues. Proximal anchor structure 120 may be a helical screw similar to distal anchor structure 110, but sized to penetrate the distance from the right atrial wall into the desired tissue. Other constructions of anchor structure 120 are also possible.

After proximal anchor structure 120 has been implanted as described above, catheter 230 is removed from the patient, again leaving a small screw head projecting into right atrium 30. A second band 122 (similar to first band 112) extends out of the patient's body from this screw head.

The two bands 112 and 122 that extend out of the patient are snared through a cinching catheter 240 (FIG. 8). Cinching catheter 240 is advanced to a location just distal of proximal anchor structure 120. The distal end of catheter 240 contains cinching structure 130 having teeth or other structures that permit cinching structure 130 to move distally along bands 112 and 122, but that can be used to lock bands 112 and 122 together (especially to prevent cinching structure 130 from moving proximally back along the bands).

When cinching structure 130 is in a position like that shown in FIG. 8, one or both of bands 112 and 122 can be pulled proximally (e.g., from outside the patient) to draw anchor structures 110 and 120 toward one another. As mentioned earlier, in the embodiment being discussed (in which anchor structure 120 is secured in a relatively rigid part of the heart) anchor structure 110 and the tissue in which it is implanted tend to move toward structure 120 more than structure 120 moves toward structure 110. When the distance between anchor structures 110 and 120 has been reduced to the desired degree, cinching structure 130 cooperates with bands 112 and 122 to prevent structures 110 and 120 from moving apart again. This may be either an inherent capability of cinching structure 130, or it may be the result of selective operation of structure 130 via catheter 240. An additional feature of the apparatus may be the ability of cinching structure 130 to allow selective reversal of its operation. For example, if the distance between structures 110 and 120 is initially decreased by too much, cinching structure 130 may be operable to release one or both of bands 112 and 122 so that the distance between structures 110 and 120 can be somewhat increased again.

The above-described movement of anchor structures 110 and 120 toward one another (especially the movement of structure 110 toward structure 120 in this embodiment) reduces the posterior annulus arc length. This reduces the area of all three posterior leaflet segments P1-P3, the anterior-posterior dimension AP (FIG. 1), and the commissure to commissure dimension. When the appropriate amount of cinching is achieved (which may be determined using fluoroscopy, echo, or other suitable diagnostic tools), cinching mechanism 130 is released from catheter 240 and the catheter is removed.

As has been said, placement of proximal anchor structure 120 at or below the average level of mitral valve annulus 56 increases cinching efficiency of the annulus. (Again, by “cinching efficiency” it is meant that a given cinching amount on the device produces maximum or near maximum effect on the mitral valve.) By placing distal anchor structure 110 in coronary sinus 40 (above mitral annulus 56 (see FIG. 4)) and proximal anchor structure 120 in the right atrium (at or below annulus 56), the average cinching plane is at or very close to the level of the mitral annulus. Therefore, cinching distal and proximal anchors 110 and 120 together more efficiently cinches mitral annulus 56, rather than possibly creating an atrial stenosis above the valve (see FIG. 9, which illustrates this less desirable condition). (In FIG. 9, reference number 250 is used for the implanted shaping element to avoid any implication that the present invention produces a condition like that shown in FIG. 9.) Even if distal anchor 110 is placed down in a tributary to coronary sinus 40, the sinus itself tends to be above the level of annulus 56. So it can still be beneficial to cinching efficiency at the level of annulus 56 to also have proximal anchor 120 below the level of annulus 56 so that the net effect of the apparatus along its entire length between anchors 110 and 120 is closer to the level of the annulus.

Additionally, placement of both distal and proximal anchor structures 110 and 120 proximal to the center of the P2 segment avoids impingement on the circumflex artery system. The circumflex/coronary-sinus crossover point typically occurs in the P1 segment or even in the P2 segment near the P1/P2 junction. Placement of anchors 110 and 120 away from the circumflex, and cinching primarily proximal to the circumflex, avoids circumflex impingement.

As a final aspect of the illustrative procedure being described (shown in FIG. 10), bands 112 and 122 are snared through a cutting catheter 260, and that catheter is advanced into contact with or proximity to cinching structure 130. Catheter 260 has a blade mechanism 262 near its distal end that is used to cut bands 112 and 122 at a prescribed distance from cinching structure 130. FIG. 10 shows conditions just after bands 112 and 122 have been cut by blade mechanism 262. Cutting catheter 260 is then removed from the patient. The patient's condition is now as shown in FIG. 1 and may be described as having (at most) only the head 144 of distal anchor structure 110 protruding into coronary sinus 40, the head 144 (at most) of proximal anchor structure 120 protruding into right atrium 30, and a band 112/122 passing between the anchors and locked into position with cinching structure 130.

Anchor structures 110 and 120 may be constructed of nitinol, stainless steel, MP35N, titanium, PEEK, cobalt chromium, or other metal or polymer compositions commonly used in medical implants. Bands 112 and 122 can be integrated into or around structures 110 and 120, respectively. While many anchor designs are possible (additional examples being described later in this specification), a particularly desirable embodiment is a helical coil. Among the advantages of such an embodiment is that a small penetration hole is created (requiring only a relatively small insertion force), while a large surface area is anchored (with a large force being required for removal of the structure). An alternative screw embodiment is shown in FIG. 29. This embodiment is similar to the embodiment shown in FIG. 5 with the addition of several barbs 146 on helix 142. Barbs 146 project out from helix 142 and are inclined backwardly, opposite the direction in which helix 142 is screwed into tissue. Accordingly, barbs 146 do not significantly impede screwing helix 142 into tissue, but they do increase resistance of helix 142 to coming out of tissue. Any of the anchor structures shown and described herein may have other features to increase surface area (e.g., surface roughness or porosity) to promote tissue in-growth and thereby increase holding power. Alternatives or additions that can be used to promote tissue in-growth include appropriate coatings and/or drugs on any of the anchor structures shown and described herein. Other features that can be employed to enhance anchoring and holding are barbs and/or glue on flexible members like 112 and 122. Such features bite into and/or engage the tissue between anchor structures 110 and 120 to help distribute anchoring load. Now elements like 112 and 122 become both linking and secondary anchoring structures. Features like these can be added to any of the linking structures shown and described herein.

An alternative or additional embodiment is shown in FIG. 11. This embodiment includes anchoring distally near the P1/P2 junction (anchor structure 110) and anchoring proximally near the P2/P3 junction (anchor structure 120). As in the previously described embodiment, cinching structure 130 cooperates with bands 112 and 122 to hold anchor structures 110 and 120 together (after they have been implanted and then pulled toward one another to the desired degree). In this embodiment both the distal and proximal anchor structures 110 and 120 are delivered into coronary sinus 40 and implanted at desired locations therein (or in a diagonal or diagonals branching off the coronary sinus as further described below (such diagonals are also elsewhere referred to herein as tributaries)). Both anchor structures 110 and 120 are driven through the wall of the coronary sinus (or diagonal(s) thereof) into mitral annulus 56, ventricular myocardium 62 (FIG. 4), or across the atrial wall into left atrium 20. Other possibilities include the use of non-penetrating embodiments such as are illustrated by FIG. 14 and described in more detail later in this specification. Still other possibilities include embodiments that penetrate into the pericardial space or (in the opposite direction) into the left atrium and brace against the far surface of the penetrated tissue to prevent removal. FIGS. 18 and 19 show examples of this type of anchor structure in which a portion 640 of the structure is initially axially aligned with the remainder, but which portion 640 becomes transverse to the remainder when it passes beyond a far wall of tissue that has been penetrated. Attempting to pull the anchor back out of the penetrated tissue is prevented by transverse portion 640 bearing on the far wall of that tissue.

The placement of anchor structures 110 and 120, drawing the anchor structures together, cinching using cinching structure 130, and cutting away the excess of bands 112 and 122 can all be similar to the corresponding aspects of the previously described embodiment. Because in this embodiment, one of the anchor structures 110 and 120 cannot generally be placed within coronary sinus 40 at or below the level of mitral annulus 56 to achieve maximum cinching efficiency (as in the previously described embodiment), one or both of structures 110/120 may be placed in a diagonal branching off the coronary sinus. However, the anatomy of these branches is highly variable and may only be usable in a subset of patients.

The just-described cinching of the P2 segment may be a stand-alone procedure, or it may be used as an adjunct to P3 segment cinching in cases where additional posterior mitral annulus arc length reduction is required to seal mitral valve 50. FIG. 12 shows an illustrative embodiment in which two sets of elements 110/112/120/122/130 have been implanted (the set with reference number suffix a typically being installed before the set with reference number suffix b).

Another alternative or additional embodiment is shown in FIG. 13. This embodiment includes anchoring distally (using anchor structure 110) near the distal knee 44 of coronary sinus 40, or even slightly down great cardiac vein 46 to be at or below the level of mitral valve annulus 56. Proximal anchor structure 120 is then placed near the P1/P2 junction, and a substantially straight segment along P1 is cinched (i.e., by drawing one or both of bands 112 and 122 proximally through cinching structure 130 and then allowing or operating structure 130 to cinch these bands when the desired amount of cinching has been achieved).

Circumflex artery 48 typically crosses coronary sinus 40 somewhere around the P1 segment of the valve, causing the placement of the distal (110) and proximal (120) anchor structures to vary from patient to patient. If the cross-over point is very distal (near trigone 59b), both anchors 110 and 120 may be placed proximal to the cross-over point. On the other hand, if the cross-over point occurs more proximally, both anchors 110 and 120 may be placed distal to the circumflex artery, with distal anchor 110 placed slightly down great cardiac vein 46.

Cinching of the P1 segment (e.g., as shown in FIG. 13) may be a stand-alone procedure. Alternatively it may be an addition to cinching another segment such as the P3 segment in cases where additional posterior mitral annulus arc length reduction is required to help valve 50 close and seal properly.

As mentioned earlier, an illustrative anchoring structure 110 or 120 comprises a helical coil screw as shown in FIG. 5, where the helix 142 is sized to penetrate through to mitral annulus 56 and/or other relatively strong tissue, while the head 144 of the screw may remain in coronary sinus 40 (or right atrium 30, or a diagonal branching off of coronary sinus 40, or the great cardiac vein, depending on the anatomy in which the anchor structure is used). The distance from the bottom of coronary sinus 40 to mitral annulus 56 varies along the length of the coronary sinus. For example, this distance may vary from more than 15 mm to less than 1 mm. The length of each screw 142/144 is preferably sized for each location to penetrate into mitral annulus 56 and/or other relatively strong tissue. Individually placed helical anchors 142/144 can be positioned according to the anatomy and spacing between coronary sinus 40 and mitral annulus 56 to take into account such highly variable anatomy. The pitch and diameter of helix 142, as well as the cross-sectional dimensions, are sized to produce secure holding force in the tissue. The cross-sectional dimensions may taper along the length of the screw 142 to provide for easier penetration with stronger holding force. Additionally, helix 142 may be tapered to provide easier insertion force.

The head 144 of the helical screw may provide a pledgeting force against the wall of sinus 40 (or whatever other tissue structure the screw head engages), and may include an additional fabric, metal, or polymer pledget (not shown). As was mentioned earlier, the surface area of the screw may be increased by roughening or porosity to promote tissue in-growth and thereby further increase resistance to coming out of the tissue. Coatings and/or drugs may be used for similar purposes. The head 144 of the screw may have slots, one or more recesses, and/or one or more protrusions, or may otherwise be shaped for engagement with a driving structure (e.g., a driving collar) on the catheter shaft (e.g., catheter 220 in FIG. 3 or catheter 230 in FIG. 7).

Alternative anchor structures 310 and 320 are shown in FIG. 14. Each of these anchor structures comprises a self-expanding or balloon-expandable stent-like structure. The expanded stent diameter is sized to be slightly larger than the diameter of coronary sinus 40 (or other tubular body conduit in which the structure will be coaxially implanted), such that the stent slightly embeds into the coronary sinus or other receiving conduit wall. Additionally, the stent structure may have one or several barbs (e.g., 340 in FIG. 15) that penetrate through the coronary sinus or other receiving conduit wall, preferably to reach suitably strong tissue to help provide firm retention of the anchor structure. For this purpose particular angular location or locations of one or more barbs like 340 may be selected to help ensure that the barb(s) will penetrate into the desired destination tissue.

The linking and cinching structures between anchor structures 310 and 320 shown in FIGS. 14 and 15 will be described in more detail later in this specification. Here it will just be briefly mentioned that these structures include a ring 324 on structure 320 through which an elongated toothed structure 314 (from structure 310) passes. Elements 314 and 324 cooperate with one another somewhat like a pawl and ratchet combination (element 324 is like a pawl, and element 314 is like a ratchet). Another way to describe elements 314 and 324 is simply as a ratchet structure, or as complementary ratchet structures. Elements 314 and 324 allow structures 310 and 320 to be drawn toward one another, but not to move away from one another. This is so because the teeth 316 on structure 314 can pass through ring 324 moving from right to left as viewed in FIG. 14, but not in the opposite direction. One side of each tooth 316 is inclined to facilitate passage through ring 324. The other side of each tooth 316 is substantially perpendicular to the longitudinal axis of structure 314 to prevent movement of the tooth back through ring 324 in the opposite direction. Teeth 316 are located on portions of structure 314 that are somewhat laterally compressible, which also helps the teeth pass through ring 324 in the direction in which they are inclined to permit such passage.

Other alternative anchor structures 410 and 420 are shown in FIG. 16. Each of these anchor structures includes an angled barb structure 440 that is formed to pierce the wall of coronary sinus 40 and/or other appropriate tissue structure(s). The opposing, inclined barb structures 440 are driven into the tissue as anchor structures 410 and 420 are cinched together. Each barb structure 440 forms an acute angle with the remainder of the associated anchor structure 410 or 420, with the apex of each such acute angle pointing generally away from the apex of the acute angle in the other anchor structure. The linking and cinching structures 414 and 424 of this embodiment are similar to the corresponding aspects 314/324 of embodiments like those shown in FIGS. 14 and 15. Teeth 416 are like above-described teeth 316. The barb 440 angles and lengths are sized to penetrate the coronary sinus wall into mitral annulus 56 (assuming such placement of the associated anchor structure 410/420) or any of the above-mentioned tissues or combinations of tissues. The angled barb design may be generally similar to the above-described barbed stent design (FIG. 15), but leaves less implanted structure in the coronary sinus. It will be understood that the proximal and distal anchors do not have to be of the same design in all cases, but that any combination of different anchor designs can be used proximally and distally as desired.

Another illustrative embodiment of an anchor structure 510/520 is shown in FIG. 17. This type of anchor structure can be used as either a distal anchor structure 510 or a proximal anchor structure 520. If a pair of such structures is used, each structure is oriented so that the barbs 540 on both structures point generally toward one another (e.g., as in the case of barbs 440 in FIG. 16). The portion 519 of each structure 510/520 may serve as an anchor point for a connecting band (not shown, but similar to band 112 or 122 in FIG. 1) that, in use, extends toward the other structure 510/520 so that a cinching structure (like 130 in FIG. 1) can be used to cinch the structures 510/520 together via their bands. Alternatively, other types of linking and cinching structures (like those shown, for example, in FIGS. 14-16) can be used between anchor structures 510/520. (Indeed, as a general matter, any of the various linking and cinching structures shown and described herein can be used with any of the anchor structure shown and described.) The wing-like elements 518 on structures 510/520 provide apposition against the upper wall of the coronary sinus (or other related conduit) to push the barb 540 against the bottom (or opposite) wall of the conduit and assist penetration of tissue during cinching. Additionally, wings 518 help to keep the coronary sinus (or other related conduit) patent during and after cinching. Wings 518 do not penetrate the wall of the surrounding conduit, but simply contact and slide along the wall to create an opposing force.

Yet another illustrative embodiment of anchor structures 610 and 620 is shown in FIGS. 18 and 19. In this embodiment the tip 640 of each anchor structure 610/620 is held planar with the longitudinal axis of the associated structure (e.g., 614) as the anchor penetrates into tissue. After penetrating into the tissue a prescribed distance, the tip 640 of the anchor flips orthogonal to the body of the anchor, thereby forming a very secure anchor in the tissue. For example, FIG. 19a shows illustrative deployment of an anchor structure 610 or 620 of the type shown in FIGS. 18 and 19. Prior to what is shown in FIG. 19a , delivery catheter 220 or 230 has been introduced into coronary sinus 40 in a relatively straight condition. A steering mechanism (e.g., a pull wire, not shown) in catheter 220 or 230 is then used to deflect the distal portion of the catheter toward the desired side of the coronary sinus as shown. Anchor structure 610 or 620 is then pushed out the distal end of catheter 220 or 230 with the end portion 640 of the anchor structure parallel to the longitudinal axis of the remainder of structure 610/620. After the distal portion of structure 610/620 has passed through the wall of coronary sinus 40 and other tissue structures X and Y, end portion 640 is sufficiently free of other constraints to flip out (as it may be resiliently biased to do) transverse to the longitudinal axis of the remainder of structure 610/620. In this transverse condition, end portion 640 very strongly resists withdrawal of structure 610/620 from the tissue. In the particular example shown in FIG. 19a, the lower surface of tissue Y may be the inner surface of the left atrium or the inner surface of the pericardial space (i.e., the epicardial surface). End portion 640 bears on that tissue surface to prevent withdrawal of structure 610/620. In other embodiments, end portion 640 may not pass all the way through tissue, but may become transverse to the remainder of structure 610/620 while embedded in tissue.

The linking and cinching structures of the FIG. 18/19 embodiment can be similar to those for the embodiment shown in FIG. 16, for example. The embodiment of FIGS. 18 and 19 is characterized by a very high removal to insertion force ratio, and is advantageous for penetrating through the atrial wall and anchoring against the inner wall of the left atrium.

Although various cinching structures have already been shown and described in some detail, some additional aspects of such structures will now be considered.

In general, the cinching structure is designed to allow shortening of the distance between each pair of distal and proximal anchor structures, and to then lock the anchor structures with this shortened distance between them. The cinching structure is preferably reversible, and is also preferably actuated percutaneously (i.e., from a control location that is outside the patient's body and via apparatus that extends from that control location through vasculature of the patient to the location of the cinching structure). The various cinching structures shown and described herein can be used in any of many different combinations with the various anchor structures also shown herein.

An illustrative embodiment of a particularly preferred cinching structure 730 is shown in FIG. 20. For example, this type of cinching structure can be used as element 130 in embodiments such as are illustrated by FIG. 1. Cinching structure 730 has a tubular structure 732 from which teeth 734 are resiliently biased to angle inwardly as shown in FIG. 20. Structure 730 is initially mounted around an inner tube (not shown) with an open, distal, free end of that tube toward the left as viewed in FIG. 20. In use, bands 112 and 122 enter the distal free end of the inner tube from the associated anchor structures 110 and 120, respectively. The ring portion 732 of structure 730 is near the distal end of the inner tube so that it is also around bands 112 and 122. Bands 112 and 122 are pulled proximally into structure 730 and the inner tube until the desired amount of cinching has been achieved. Structure 730 is then held in place (e.g., by an outer tube (not shown) cooperating with other features 736 on structure 730) while the inner tube is retracted proximally. This eventually pulls inner tube out from inside prongs or teeth 734, which allows the prongs to spring inwardly into firm engagement with bands 112 and 122. This secures bands 112 and 122 together. Structure 730 can then be released from the above mentioned outer tube and the outer tube can also be withdrawn proximally. If sufficient cinching is not achieved at first, more cinching can be achieved by pushing on structure 732 (e.g., via features 736) while pulling proximally on bands 112 and 122. Even without the above mentioned inner tube being present, structure 730 can be shifted distally along bands 112 and 122 to increase the amount of cinching effected. Typically, fluoroscopy or echocardiography is used to diagnose mitral valve performance and the appropriate amount of cinching to seal the valve.

Another illustrative embodiment of a cinching structure 830 is shown in FIG. 21. This embodiment comprises a helical coil spring-like structure that is held elongated in tension to allow each band 112 and 122 to enter the coil between two adjacent turns, to pass axially along the inside of the coil for some distance, and to then exit the coil between two other adjacent turns. As long as structure 830 is thus elongated, the structure can move axially relative to bands 112 and 122. When a desired amount of cinching has been produced, structure 830 is removed from tension. This takes away the spacing between adjacent turns of the coil and locks both bands 112 and 122 to structure 830 and thus to one another. This design is reversible in that tension can be alternately applied and released to allow movement of structure 830 in either direction along bands 112 and 122 or to provide locking of the bands. Features 832 and 834 at axially opposite ends of structure 830 can be used to selectively apply tension and elongation to structure 830 (e.g., by respectively cooperating with two coaxial catheter-like elements that are axially movable relative to one another).

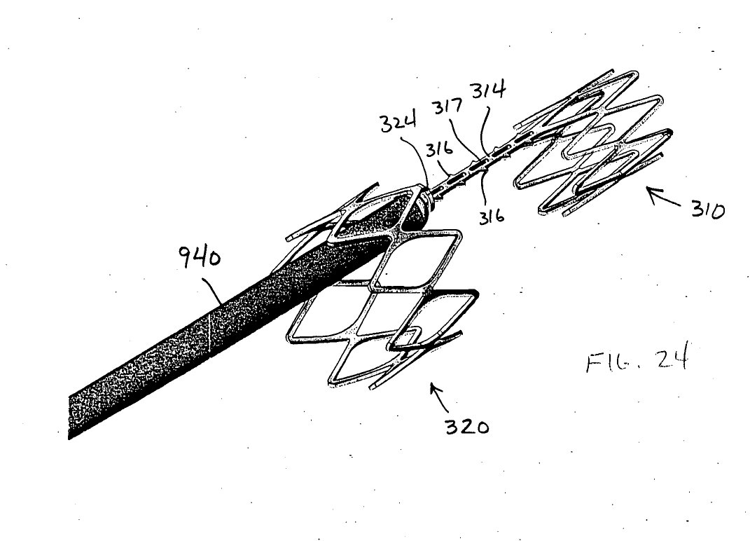

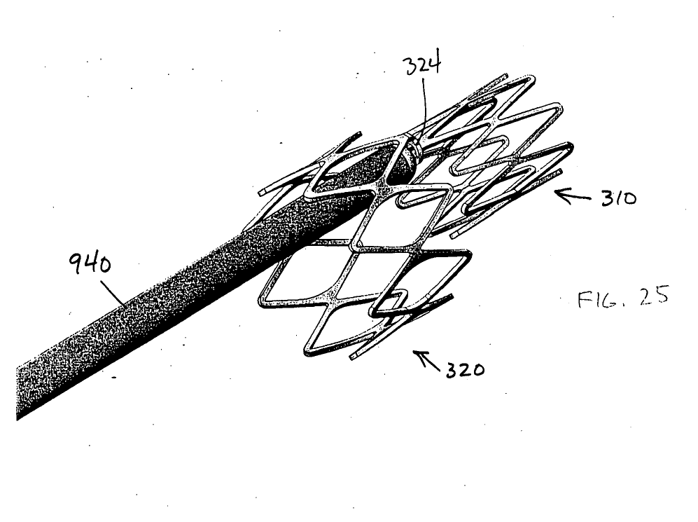

Yet another illustrative embodiment of cinching apparatus is shown in FIGS. 22-27. These FIGS. illustrate this embodiment in a context like that shown in FIG. 14, and so reference numbers from FIG. 14 are used again in FIGS. 22-27.

FIG. 22 shows that a portion 314 of this cinching structure projects from distal anchor structure 310 as a series of protrusions 316 and slots 317. A second portion of this cinching structure extends from proximal anchor structure 320 in the form of an eyelet 324. As shown progressively in FIGS. 2325, hollow cinching catheter 940 can be used to push eyelet 324 distally toward and then distally along structure 314 until a desired amount of shortening of the distance between anchor structures 310 and 320 has been achieved.

To facilitate alignment and initial mating of structures 314 and 324, a flexible band (e.g., a length of suture material) may extend proximally from the proximal end of structure 314 all the way out of the patient when anchor structure 310 is first implanted. Anchor structure 320 may then be introduced into and implanted in the patient with eyelet 324 around this flexible band. Thereafter, cinching catheter 940 may be introduced into the patient with a lumen of the catheter around this flexible band. Some tension on this flexible band helps the distal end of catheter 940 align with eyelet 324, and then helps eyelet 324 align with structure 314. The ability to pull proximally on the flexible band while pushing distally on cinching catheter 940 helps to draw anchor structures 310 and 320 toward one another as cinching proceeds (shown progressively in FIGS. 23-25). At the end of the cinching process, a cutter catheter (e.g., like catheter 260 in FIG. 10) may be used to cut away and remove a portion of the above-mentioned flexible band.

As eyelet 324 passes over each transversely adjacent pair of protrusions 316, the material around the slot 317 between those protrusions may deflect inwardly. This helps the protrusions pass through eyelet 324. Protrusion 316 are shaped to allow their passage through eyelet 324 in the direction associated with movement of structures 310 and 320 toward one another, but to strongly resist passage through eyelet 324 in the opposite direction. However, passage in the opposite direction may be achievable (e.g., to reduce the amount of cinching that has been achieved) by providing catheter 940 or other similar apparatus with the ability to selectively squeeze structure 314 together adjacent the protrusions that are currently engaging eyelet 324. When thus squeezed toward one another, these protrusions 316 can slip back through eyelet 324 to reverse some previously effected cinching. This squeezing to reduce cinching can be repeated as many times as necessary to release the desired amount. Protrusions 316 can be spaced in prescribed increments to provide a controlled, measurable amount of cinching each time a pair of protrusions passes through eyelet 324. For example, each such successive “click” (i.e., passage of a pair of protrusions 316 through eyelet 324) may give 2 mm of cinching.

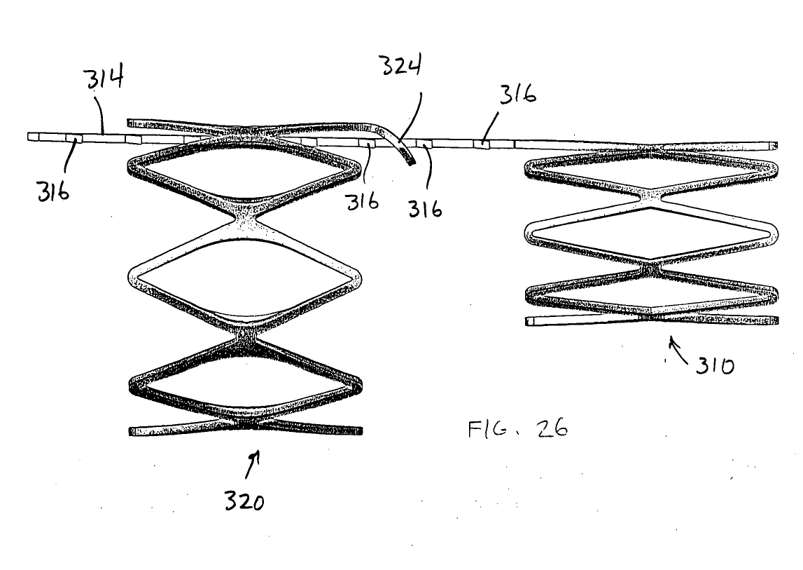

The fully cinched structure is shown in FIG. 14, and alternatively in FIGS. 26 and 27. Comparison of these FIGS. illustrates the point that cinching can stop after all of protrusions 316 have passed through eyelet 324 (FIG. 14) or after only some of protrusions 316 have passed through eyelet 324 (FIGS. 26 and 27).

Another illustrative embodiment of an anchor structure 230/1020 is shown in FIGS. 30-34. This embodiment may be described as a helical screw with an internal T-shaped anchor and compressive pledgeting. This structure combines some features of previously described helical screw anchors (e.g., as in FIG. 5) and T-shaped anchors (e.g., as in FIGS. 18 and 19). This type of combined anchor structure can be used to produce a holding force in the tissue, both acutely and chronically, that is greater than either of its constituents can produce alone. The increase in acute holding force results from a greater anchor-to-tissue surface area in the direction of applied force, as well as the effect of compressing the tissue between the pledget and the transverse portion of the T-shaped anchor. The chronic holding force increase results from a greater total surface area of the pledget, helical screw, and T-shaped anchor combination.

The deployment of a combination helical screw and internal T-shaped anchor with pledget is shown in FIGS. 30-34. FIG. 30 shows an outer guide catheter 1103 positioned against the wall of a desired target tissue region 1100. A second internal catheter 1102 contains a helical screw 123 mounted at its distal end. Screw 123 is attached to catheter 1102 by a key-type mechanism 1104, which allows the catheter to transmit axial and torsional force to the screw. As shown, internal catheter 1102 has been turned the appropriate amount of revolutions to drive screw 123 into tissue region 1100 until the head of the screw has bottomed out on the tissue wall. The resulting catheter/screw combination provides an adequate guiding and apposition platform for subsequent steps of driving another anchor through the lumen of screw 123.

FIGS. 31-34 show how the T-shaped anchor is deployed through the lumen of screw 123. A third internal catheter 1108 is advanced through the lumen of second catheter 1102. Catheter 1108 has a washer-type pledget 1107 attached to its distal tip as shown in FIG. 31. Simultaneously, a fourth internal catheter 1109 is advanced along with third catheter 1108. Catheter 1109 contains a cord 1110 (e.g., of dacron) in its lumen. Cord 1110 is attached to a T-shaped anchor 1105 at the distal end of catheter 1109. This distal tip of catheter 1109 is used to push T-shaped anchor 1105 axially through the lumen of catheters 1102 and 1108. T-shaped anchor 1105 includes a distal portion that is constrained axially until released, after which the constrained portion flips out to a transverse position. The distal tip of anchor 1105 is sharp so that it can pierce tissue. The proximal portion of anchor 1105 attaches to cord 1110 and contains a set of angled struts 1106.

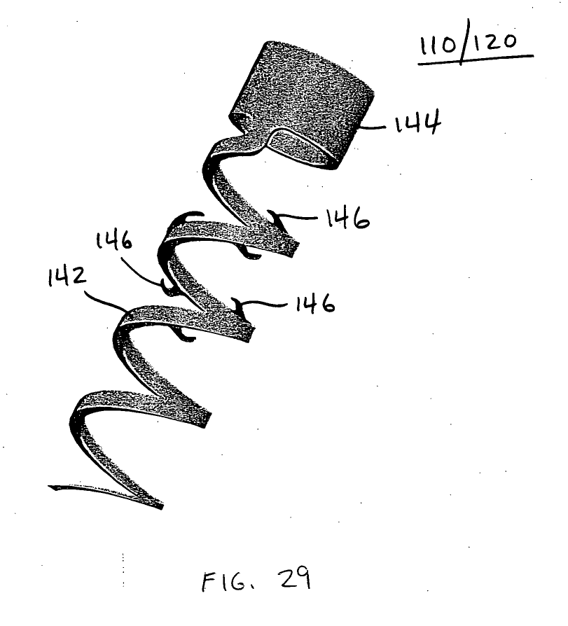

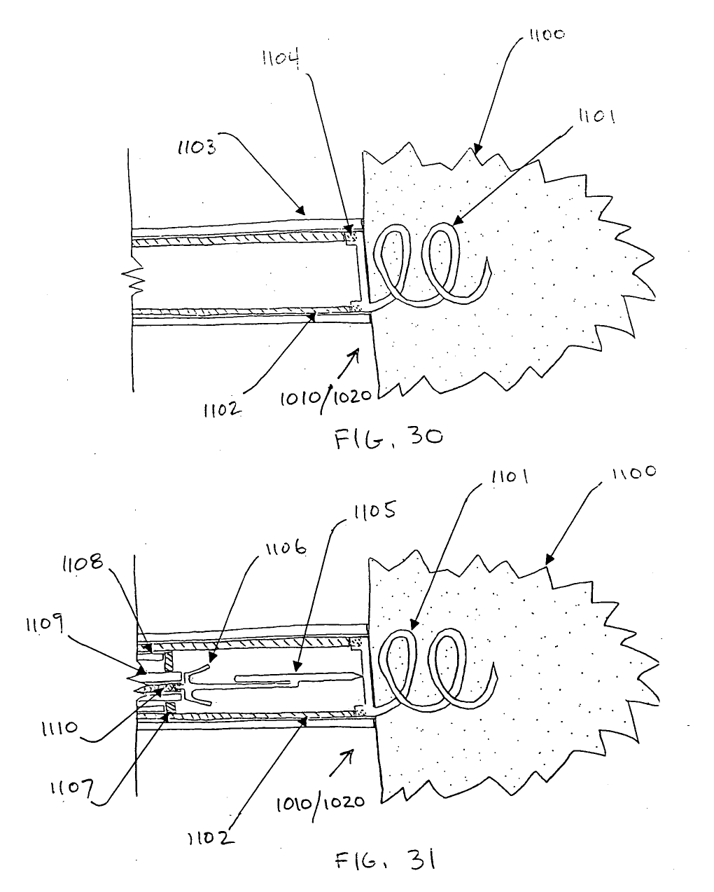

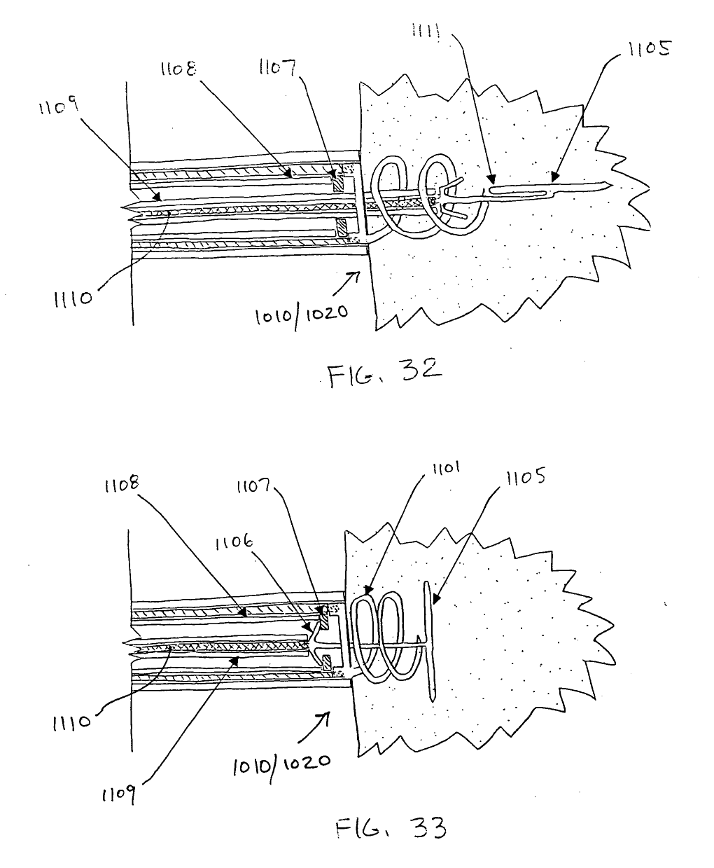

T-shaped anchor 1105 is deployed by advancing catheter 1109 distally so that the T-shaped anchor pierces through the tissue and pushes through the lumen of helical screw 123 until the constrained portion of anchor 1105 is all the way through the screw as shown in FIG. 32. Simultaneously, catheter 1108 is pushed distally until pledget 1107 has bottomed out on the head of screw 123. The constrained portion of T-shaped anchor 1105 is released by pulling cord 1110 (and hence anchor 1105) proximally. T-shaped anchor 1105 is designed to pierce one-way (i.e., distally) through the tissue. If anchor 1105 is pulled proximally, the proximal edge 1111 of the constrained portion catches on the tissue and releases the constrained portion, thereby causing it to flip out transversely to the axis of helical screw 123. Once anchor 1105 flips out transversely, it provides a large surface area to gather tissue, which prevents the anchor from pulling out.

Once anchor 1105 has flipped transversely, catheter 1109 and cord 1110 are pulled proximally until the angled flexible struts 1106 are pulled through pledget 1107. The ends of struts 1106 are at a larger diameter than the internal diameter of pledget 1107. Because struts 1106 are flexible and angled, they compress to a smaller diameter while being pulled through pledget 1107. Struts 1106 expand outwardly when their tips reach the proximal side of pledget 1107 (see FIG. 33). The distance between the transverse portion of anchor 1105 and struts 1106 is designed so that helical screw 123 is put in compression when struts 1106 lock to the proximal side of pledget 1107. Finally, as shown in FIG. 34, the catheters are removed, leaving the resulting anchor combination behind with cord 1110 extending out of the patient's body.

The parts of the anchor shown in FIGS. 30-34 may be fabricated from nitinol, stainless steel, or any other biocompatible metal or polymer. Each piece may be fabricated from a different material. One or multiple pieces may be covered with fabric such as dacron to promote and accelerate healing and in-growth of tissue around the implant to increase the holding force of the anchors.

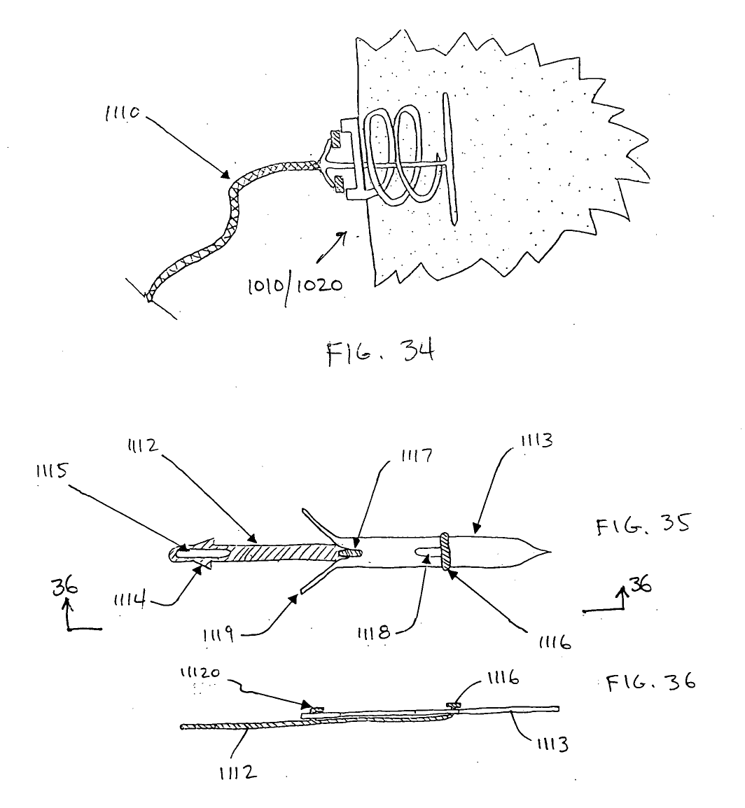

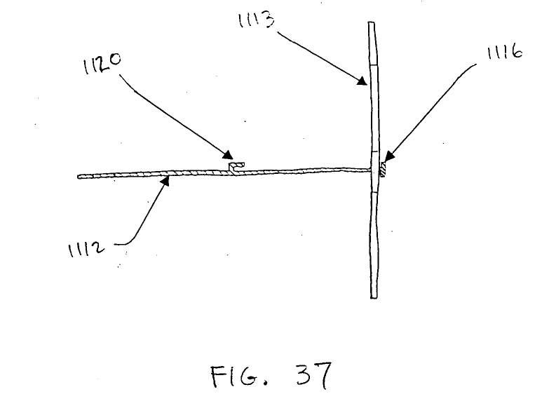

An illustrative embodiment of a T-shaped anchor structure (e.g., for use as the T-shaped portion of the anchor structure shown in FIGS. 30-34) is shown in FIGS. 35-37. The portion that flips out transversely as described above may be a separate piece 1113 that contains a sharp distal tip, a slot 1118 for accepting an interlocking member 1116, a proximal end with a U-shaped notch 1117 for accepting a second interlocking member 1120, and a pair of flexible angled struts 1119 for catching tissue when pulled proximally.

The second piece 1112 of the T-shaped anchor connects to the first piece, locks onto the pledget, and attaches to the dacron cord as described in connection with FIGS. 30-34. The proximal end also includes a pair of angled struts 1114 that compress inward when pulled through the pledget and expand outward and lock on the back of the pledget once they have made it all the way through. The distal end of the second piece includes a T-shaped feature 1116 that interlocks with the slot 1118 in the first piece. Slot 1118 is longer than it is wide so that the first piece can move axially relative to the second piece. Also, the second piece includes a limiter 1120 that is formed in a L-shape.

The T-shaped anchor is loaded for deployment by constraining the U-shaped notch 1117 in the proximal end of the first piece under the limiter 1120 of the second piece. In doing so, the T-shaped feature 1116 is bent, which puts the first piece under load, creating a bias for flipping the first piece out transversely once the trap is sprung. When loaded, the two pieces will remain locked when a force is applied that pushes the two pieces toward each other, as in the case when driving and piercing the anchor into tissue through the lumen of the helical screw as described in connection with FIGS. 30-34. But when a force is applied in the opposite direction, such as when the dacron cord is pulled proximally after the anchor has been driven through the tissue, the angled struts 1119 catch on tissue, which results in the first piece being pulled away from the second piece. This allows U-shaped feature 1117 to move out from under limiter 1120, and the trap is sprung, causing first piece 1113 to flip out transversely to the axis of second piece 1112 as shown in FIG. 37.

Once again, the pieces of the anchor structure shown in FIGS. 35-37 may be fabricated from nitinol, stainless steel, or any other biocompatible metal or polymer. Each piece may be fabricated from a different material. The pieces may be covered with a fabric such as dacron to promote and accelerate healing and in-growth of tissue around the implant to increase the holding force of the anchors.

It is within the scope of the invention that variations of the preceding embodiments are possible and that certain steps or pieces may be omitted, or the order of operations may be modified without departing from the spirit of the invention. For example, the pledget 1107 in FIGS. 30-34 may be omitted, whereby the spring 123 is not put under compression, and all that is left for an implant is the helical screw and the T-shaped anchor. In another scenario, the helical screw may only be used as a mechanism to provide adequate apposition, or back-up support, in order to drive a T-shaped anchor into the tissue, whereby the screw would be unscrewed and removed after the T-shaped anchor was deployed, leaving only the T-shaped anchor as the implant. In still another scenario, the T-shaped anchor may be driven all the way through a wall of the heart into an open plenum such as the left atrium, or the pericardial space outside the heart. This may be especially effective for driving T-shaped anchors through the papillary muscles out into the pericardial space outside the heart in order to remodel the left ventricle by tethering and drawing the papillary muscles closer together, thereby fixing an insufficient mitral valve, as well as remodeling a dilated ventricle. This last point will be considered in more detail later in this specification.

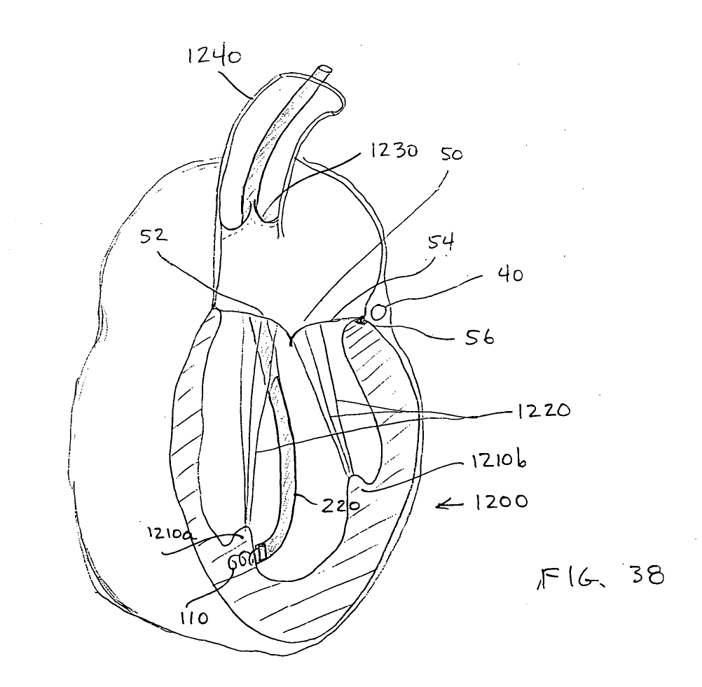

Another example of a possible use of the invention is for remodeling the left ventricle of the heart as a treatment for congestive heart failure and/or repairing an insufficient mitral valve. FIG. 38 is generally similar to FIG. 4, but with somewhat more attention given to left ventricle 1200. Previously mentioned features are mitral valve 50 (with its anterior leaflet 52, posterior leaflet 54, and annulus 56) and coronary sinus 40. Papillary muscle regions 1210a and 1210b are shown in the lower portion of left ventricle 1200. Also shown are the cordae tendenae 1220 that extend up from papillary muscle 1210 to the leaflets 52 and 54 of mitral valve 50. Aortic valve 1230 and aorta 1240 connect to the upper portion of left ventricle 1200.

A well known type of heart disease is enlargement of the left ventricle. Among the possible consequences of such left ventricle enlargement is an inability of the mitral valve to close or seal properly because it is held partly open by the cordae tendenae 1220 extending from the displaced papillary muscle region 1210.

The present invention can be used to remodel an enlarged left ventricle in order to reduce its size. This improves heart function and also allows the mitral valve to function adequately again.

FIG. 38 shows a first anchor structure 110 being implanted in the wall of left ventricle 1200 from a delivery catheter 220 in accordance with the invention. Catheter 220 and other subsequently used catheters may be introduced into left ventricle 1200 via aorta 1240 and aortic valve 1230. Thus this left ventricle remodeling procedure may be performed percutaneously if desired. Any of the anchor structures shown and described herein can be used for anchor structure 110. A good location for anchor structure 110 may be in papillary muscle region 1210a below the attachment point for the cordae tendenae 1220 extending up to anterior leaflet 52.

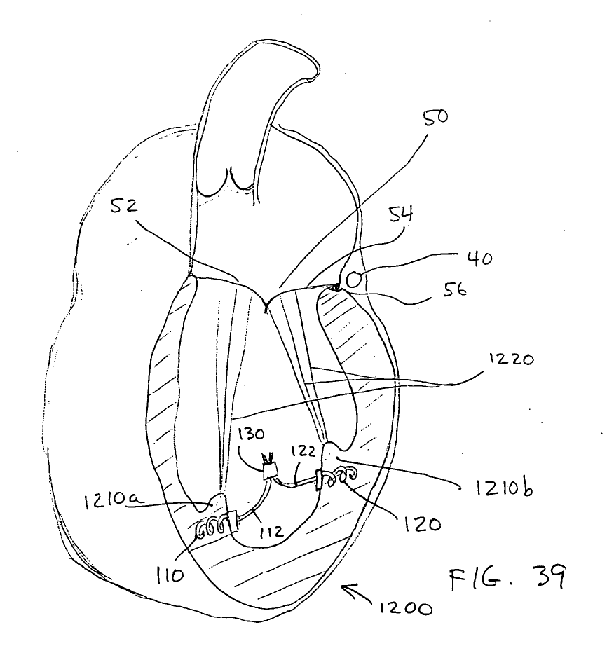

After first anchor structure 110 has been implanted as shown in FIG. 38, a second anchor structure 120 is similarly implanted on the opposite side of left ventricle 1200 as shown in FIG. 39. Again, a good location for anchor structure 120 may be in papillary muscle region 1210b below the attachment point for the cordae tendenae 1220 extending up to posterior leaflet 54.

After both of anchor structures 110 and 120 have been implanted, flexible bands 112 and 122 extending from them are pulled proximally through cinching structure 130 as described earlier in this specification for other uses of the invention. This pulls anchor structures 110 and 120 and the associated tissue structures toward one another to remodel left ventricle 1200. When the desired amount of remodeling has been achieved, cinching structure 130 is operated or allowed to operate to prevent anchor structures 110 and 120 from moving apart again. The remodeling of left ventricle 1200 is thereby made permanent. Among the benefits of drawing the depicted portions of papillary muscle 1210 toward one another in the manner shown in FIG. 39 is that the cordae tendenae 1220 no longer hold mitral valve 50 open when it should close.

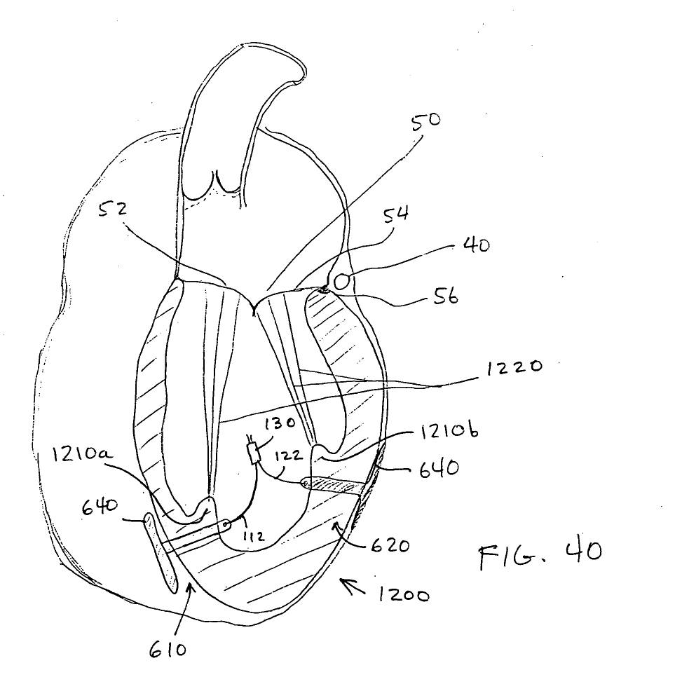

FIG. 40 shows an alternative embodiment of what is shown in FIGS. 38 and 39. FIG. 40 shows the use of T-shaped anchor structures 610/620 (e.g., like those shown in FIGS. 18 and 19 or like the T-shaped anchors or portions of anchors shown in FIGS. 30-37). FIG. 40 shows these anchor structures passing all the way through the wall of left ventricle 1200 so that the transverse portion 640 bears on the outer surface of that wall. (The corresponding transverse portions of the anchors shown in FIGS. 30-37 are portions 1105 in FIGS. 30-34 or portion 1113 in FIGS. 35-37).

In general, it will be understood that the reference numbers used for the various apparatus components shown in FIGS. 38-40 are only illustrative and are not intended as limiting. Thus, for example, any anchor structure shown herein that would be suitable can be used for elements 110 and 120 in FIGS. 38 and 39. It will also be understood that the particular locations of anchors 110, 120, 610, and 620 shown in FIGS. 38-40 are only illustrative, and that other locations can be used to produce remodeling variations. Other examples include cinching from the vicinity of annulus 56 to a papillary muscle 1210, cinching across the middle of the ventricle, cinching along the wall of the ventricle, etc.

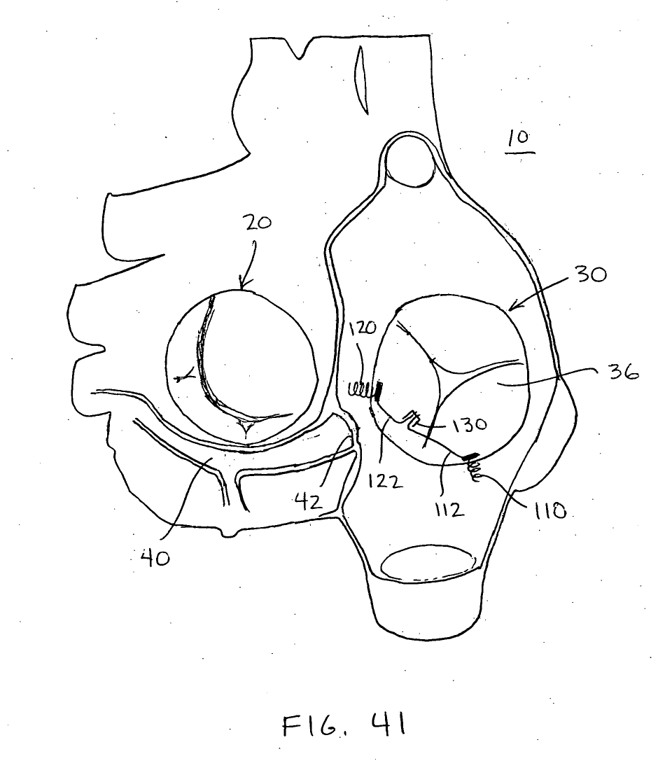

Another illustrative example of use of the invention is shown in FIG. 41. This is remodeling of right atrium 30, e.g., to improve the functioning of tricuspid valve 36. Anchor structure 110 is implanted in the wall of right atrium 30 at one location around that wall above valve 36. Anchor structure 120 is implanted in the wall of right atrium 30 at another location around that wall above valve 36. Linking structure 112/122/130 is used to pull anchor structures and the tissues in which they are implanted toward one another and to thereafter hold these structures in their new relative positions. Right atrium 30 is thereby remodelled and the performance of valve 36 is accordingly improved. Delivery of anchor structures 110 and 120 and their associated elements into right atrium 30 and operation of those elements can be similar to what has been described earlier in this specification for mitral valve repair. Thus again this right atrium remodelling may be performed percutaneously if desired.

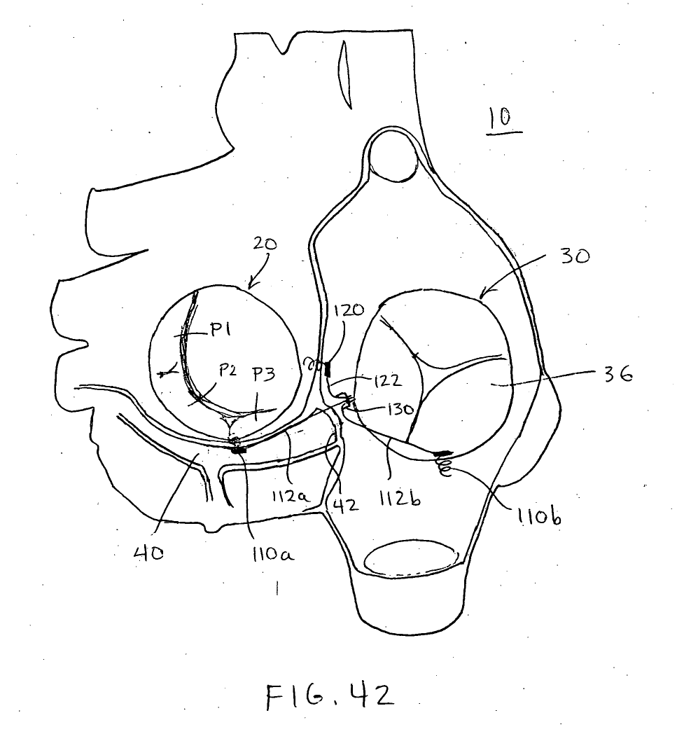

FIG. 42 shows that mitral valve repair (e.g., as in FIG. 1) can be combined with tricuspid valve repair (e.g., as in FIG. 41). Structures 110a/112a/120/122/130 perform the mitral valve repair. Structures 110b/112b/120/122/130 perform tricuspid valve repair. Thus in this illustrative embodiment, elements 120, 122, and 130 are common to both repairs.

Depending on the use being made of the invention, it may be desirable to implant one or more anchor structures in particular tissue structures. Returning, for example, to the type of mitral valve remodeling illustrated by FIG. 1, it may be one objective of the invention to anchor through the wall of coronary sinus 40 or right atrium 30 into a particular tissue structure. Ideally, anchors 110 and 120 penetrate into the fibrous tissue comprising the annulus 56 of mitral valve 50 for most efficient cinching and long-term durability. It may be desirable to deliver the anchor structures within a particular range of angular orientation about the longitudinal axis of the coronary sinus in order to achieve the best cinching efficiency and to avoid penetrating through the coronary sinus wall in a less effective direction (angular orientation).

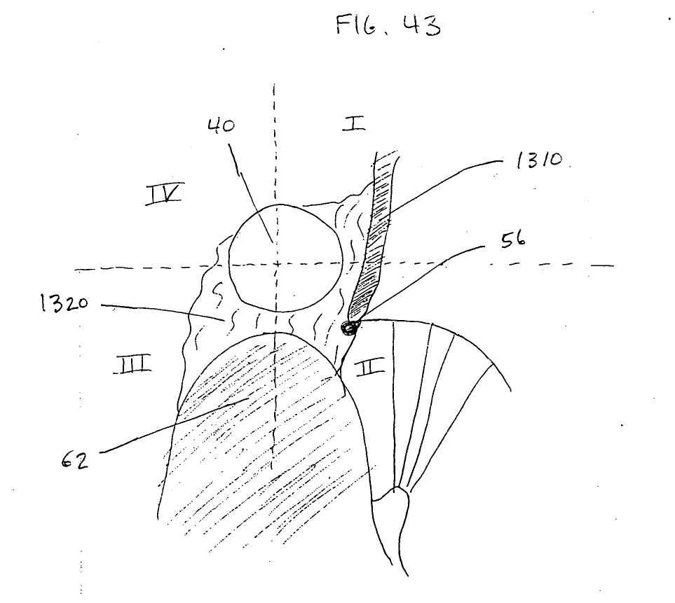

As is well known, the coronary sinus lies superior and posterior to the annulus of the mitral valve. Placement of a tissue-penetrating anchor into the coronary sinus may benefit from having a particular angular orientation about the longitudinal axis of the coronary sinus. As shown in FIG. 43, for example, a cross-sectional view of coronary sinus 40 and adjacent tissue structures can be described in terms of appropriate quadrants I-IV. The various tissue structures shown in FIG. 43 are coronary sinus 40, mitral valve annulus 56, ventricular myocardium 62, atrial myocardium 1310, and connective tissue and fat 1320.

If a penetrating anchor is deployed to the outer wall of coronary sinus 40 (quadrant IV), it may not anchor into any tissue structure providing sufficient anchoring force. If an anchor penetrates through the coronary sinus wall in quadrant I, it may penetrate through the relatively thin left atrial wall 1310. While this may be the preferred anchoring location for some embodiments, it may be more preferred to avoid leaving any foreign material in the left atrium (which may thrombose and embolise, or which may negatively interact with the mitral leaflets). If an anchor penetrates through the coronary sinus wall approximately in the region of quadrant III, it may lodge in a combination of ventricular myocardium and fat located on the outside of the AV groove. This fat/myocardium combination may provide an insufficient anchoring medium, and there may also be a possibility of puncturing small coronary arterial vessels. The most preferred region for anchor deployment is quadrant II, which tends to be the best orientation for anchoring into a combination of mitral annulus and ventricular myocardium. This provides maximum cinching efficiency (defined earlier) and durability of the implant, while avoiding the possible disadvantages mentioned above.

From the foregoing it will be seen that it is preferred to deploy one or more anchors that penetrate through the wall of coronary sinus 40 and anchor into the approximate 90° quadrant (about the longitudinal axis of the coronary sinus) defined between the plane of the mitral valve annulus and the orthogonal long-axis plane through the apex of the heart. This is approximately quadrant II in FIG. 43. More preferably, the anchor or anchors penetrate a 45° region of this quadrant that is angularly closer to the long-axis plane mentioned in the preceding sentence. The proximity of this region to the mitral annulus and the stronger fibrous tissue structure of the annulus combine to make this area likely to be best for anchoring and most efficiently cinching the mitral valve.

Placement of orientation-specific anchors into the coronary sinus or right atrium can be achieved in accordance with the invention by using a delivery catheter with specific flexibilities and a compound curvature. The flexibility of the catheter may be varied along its length, with a more rigid proximal section from the insertion site (jugular, sub-clavian, or femoral) through the superior vena vaca or inferior vena cava into the right atrium (approximately 40-70 cm). An intermediate flexibility located distal to the proximal shaft runs approximately 2-10 cm from the right atrium into the coronary sinus. A more flexible region (from 1-5 cm in length) comprises the distal-most tip of the delivery catheter.

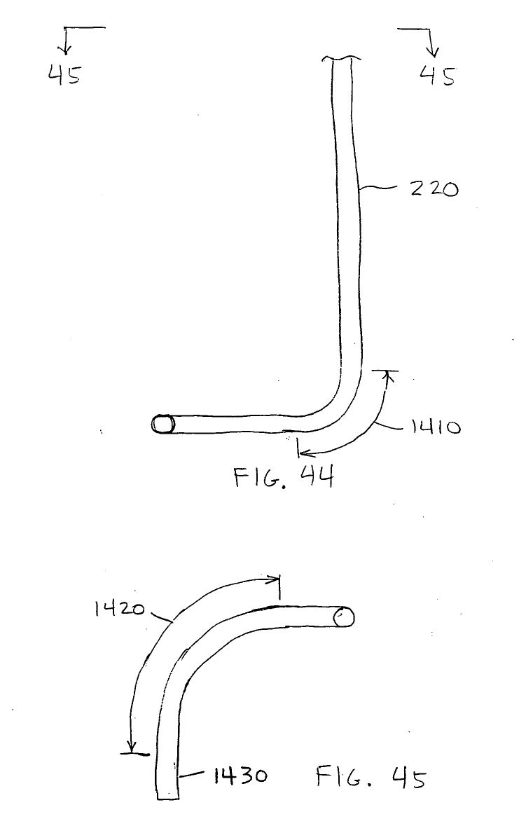

Illustrative compound curvature is formed into representative delivery catheter 220 as shown in FIGS. 44 and 45. The more proximal curve 1410 is shaped approximately to the curvature from the superior vena cava 32 into the ostium 42 of coronary sinus 40 (reference FIG. 1, for example) (or alternatively from the inferior vena cava 34 into the ostium of the coronary sinus). The secondary, more distal curvature 1420 is approximately the curvature of the coronary sinus between the ostium and the interventricular vein. The flexibility of the compound curve section of the delivery catheter 220 is preferably slightly less than that of the tissue, such that the catheter preferentially self-orients to the shape of the atrium/ostium/coronary-sinus curvature. The catheter 220 can be advanced into position with a more rigid cannulator (not shown) having a shape that is optimal to gain entrance into the coronary sinus. When the cannulator is removed from the delivery catheter 220, the catheter self-orients to the compound shape of the atrium/ostium/coronary-sinus. Once in position, a significant amount of torsion on the proximal end of the delivery catheter is required to rotate the catheter out of the “sweet spot.” Thus the delivery catheter 220 can be used to key a rotational orientation relative to the longitudinal axis of the coronary sinus 40.

The anchor structures of this invention can be delivered within the preferred region of the coronary sinus by a variety of methods keying off the above-described self-oriented delivery catheter. In a preferred embodiment, a steerable distal tip 1430 is formed on the delivery catheter 220 by placing a pull wire for the distal tip in the angular orientation about the longitudinal axis of the delivery catheter corresponding to the preferred angular orientation within the coronary sinus and about the longitudinal axis of the coronary sinus. For example, a pull wire located at 30° to the plane of the distal-most curvature 1420 on the delivery catheter will correspond to a tissue location 30° to the plane of the mitral valve. Therefore, the delivery catheter 220 can be used to deliver anchors into the preferred region around the coronary sinus circumference as described above in connection with FIG. 43.

The compound curvature illustrated by FIGS. 44 and 45 and described above may have any of the following characteristics depending, for example, on the anatomy to be served: curvature 1410 may lie in the same plane as or a different plane than curvature 1420; curvatures 1410 and 1420 may have radii of similar or different lengths; and curvatures 1410 and 1420 may have common or different centers. However, the curvatures will not be “compound” if all three of these characteristics are the same for both curvatures (i.e., if both curvatures are in the same plane with the same center and the same radial length).

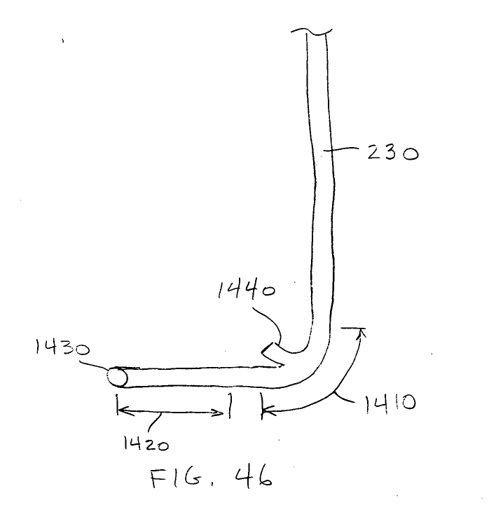

If desired, the proximal anchor 120 (e.g., FIG. 1) can be oriented within the right atrium using a similar approach. In this case delivery catheter 230 branches as shown in FIG. 46. Except for the addition of branch 1440, catheter 230 can be similar to catheter 220 in FIGS. 44 and 45. One branch (including curve 1420) passes into the coronary sinus. The other branch (1440) is directed toward the proximal anchor target area below the coronary sinus ostium.

As a possible alternative to the foregoing, other devices (e.g., balloons or expanding structures) with built-in curvature can be used to map to the curvature of the coronary sinus and provide orientation of the anchor delivery. As another example, proximal anchor structure 120 can be oriented within the right atrium by use of coronary sinus tributaries. More specifically, the tip of the delivery catheter can be lodged in the ostium of the middle cardiac vein or the small cardiac vein (located in the right atrium near the coronary sinus ostium). Lodging the distal tip of the delivery catheter locks the catheter in a specific location within the right atrium. The compound curvature of the catheter, in combination with the fixed tip location, can be used to direct anchor deployment into the location with the desired orientation.

The following is a brief recapitulation of some of the more important possible aspects of the invention. One such aspect in mitral valve repair is anchoring above and below the plane of the mitral valve annulus to effectively cinch near the average plane of the annulus. Another such aspect in mitral valve repair is anchoring (and therefore cinching) across a substantially straight segment of the coronary sinus and shortening that distance. A third such aspect in mitral valve repair is implanting the proximal anchor in tissue that is more rigidly fixed than the distal anchor. This means that the distal anchor (in more flexible posterior tissue) moves toward the proximal anchor substantially more than the proximal anchor moves toward the distal anchor. A fourth such aspect of the invention in mitral valve repair is providing a specific orientation (e.g., angularly around the longitudinal axis of the coronary sinus) of anchoring to anchor into specific tissue structures (e.g., the mitral valve annulus and/or the ventricular myocardium). A fifth such aspect relates to the various anchor, linking, and cinching structures disclosed (e.g., helical screws, “grasshopper” (e.g., as in FIG. 17), angled barbs (e.g., elements like 340 in FIG. 15, 440 in FIG. 16, 540 in FIG. 17, and 146 in FIG. 29), anchors somewhat like sheetrock screws (e.g., as in FIGS. 18 and 19), ratcheting mechanisms (e.g., like elements 314, 316, and 324 in FIG. 14, elements 414, 416, and 424 in FIG. 16, and elements 614, 616, and 624 in FIGS. 18 and 19), fabric cinching mechanisms (e.g., as in FIGS. 20 and 21), and cord cinching mechanisms (e.g., as in FIG. 21). A sixth such aspect in mitral valve repair is using compound curvature on a delivery catheter and curvature of the coronary sinus anatomy to guide orientation of anchor deployment. It will be understood that not all of these aspects (or indeed any of these aspects) may be employed in any particular embodiment of the invention.

It will be understood that the foregoing is only illustrative of the principles of the invention, and that various modifications can be made by those skilled in the art without departing from the scope and spirit of the invention.

What is claimed is:

1. A method of shortening a portion of the perimeter of a patient's mitral heart valve comprising:
introducing a first anchor structure via at least a portion of the coronary sinus of the patient's heart and securing the first anchor structure to the patient's tissue;
introducing a second anchor structure into the patient and securing the second anchor structure to the patient's tissue at a location that is in communication.with the first anchor structure;
providing a linking structure between the first and second anchor structures; and
shortening the linking structure to reduce distance between the first and second anchor structures.

2. The method defined in claim 1 wherein all of the introducing, providing, and shortening are performed percutaneously.

3. The method defined in claim 1 wherein the linking structure includes first and second flexible members extending respectively from the first and second anchor structures, and a securing structure in engagement with the flexible members and movable along at least one of the flexible members, and wherein the shortening comprises:
moving the securing structure along the at least one of the flexible members toward the anchor structure from which that flexible member extends.

4. The method defined in claim 3 further comprising:
causing the securing structure to non-movably engage both of the flexible members after the moving has been performed to a desired degree.

5. The method defined in claim 4 further comprising:
causing the securing structure to again movably engage at least one of the flexible members if it is desired to again move the securing structure opposite to the moving.

6. The method defined in clam 1 wherein the linking structure includes a ratchet connection between the first and second anchor structures, the ratchet structure permitting movement of the first and second anchor structures toward one another, but resisting movement of the first and second anchor structure away from one another, and wherein the shortening comprises:
moving the first and second anchor structures toward one another with the ratchet structure in operation to resist opposite movement of the first and second anchor structures.

7. The method defined in claim 6 wherein the ratchet structure is selectively releasable to permit movement of the first and second anchor structures away from one another, and wherein the method further comprises:
releasing the ratchet structure if it is desired to permit the first and second anchor members to move away from one another.

8. The method defined in claim 1 wherein the securing the first anchor structure disposes the first anchor structure on a first side of an average level of the mitral annulus, and wherein the securing of the second anchor structure disposes the second anchor structure on a second side of an average level of the mitral annulus.

9. The method defined in claim 1 wherein the securing the first anchor structure disposes the first anchor structure adjacent the P3/P2 junction of the mitral valve.

10. The method defined in claim 9 wherein the securing the second anchor structure disposes the second anchor structure in the right atrium.

11. The method defined in claim 10 wherein the securing the second anchor structure disposes the second anchor adjacent the ostium of the coronary sinus.

12. The method defined in claim 1 wherein the securing the first anchor structure disposes the first anchor structure adjacent the P1/P2 junction of the mitral valve.

13. The method defined in claim 12 wherein the securing the second anchor structure disposes the second anchor structure adjacent the P3/P2 junction of the mitral valve.

14. The method defined in claim 1 wherein the securing the first anchor structure disposes the first anchor structure distal of the P1/P2 junction of the mitral valve.

15. The method defined in claim 14 wherein the securing the second anchor structure disposes the second anchor structure proximal the first anchor structure and on a same side of the point at which the circumflex artery crosses the coronary sinus as the first anchor structure.

16. The method defined in claim 15 wherein the securing the first anchor structure disposes the first anchor structure distal the point at which the circumflex artery crosses the coronary sinus.

17. The method defined in claim 15 wherein the securing the first anchor structure disposes the first anchor structure proximal the point at which the circumflex artery crosses the coronary sinus.

18. The method defined in claim 1 wherein the providing includes:
disposing at least a portion of the linking structure in the coronary sinus.

19. The method defined in claim 1 wherein the securing the first anchor structure comprises:
enlarging the first anchor structure so that it substantially annularly engages a surrounding annulus of tissue.

20. The method defined in claim 1 wherein the securing the second anchor structure comprises:
enlarging the second anchor structure so that it substantially annularly engages a surrounding annulus of tissue.

21. The method defined in claim 1 wherein the securing the first anchor structure comprises:
causing a portion of the first anchor structure to penetrate tissue.

22. The method defined in claim 1 wherein the securing the second anchor structure comprises:
causing a portion of the second anchor structure to penetrate tissue.

23. Apparatus for use in shortening a portion of the perimeter of a patient's mitral heart valve comprising:
a first anchor structure adapted for percutaneous introduction via at least a portion of the coronary sinus of the patient's heart and for securement to the patient's tissue;
a second anchor structure adapted for percutaneous introduction into the patient and for securement to the patient's tissue at a location that is in communication with the first anchor structure; and
a linking structure adapted to extend between the first and second anchor structures, the linking structure being of adjustable length whereby a distance between locations at which the first and second anchor structures are secured to the patient's tissue can be reduced.

24. The apparatus defined in claim 23 further comprising:
means adapted for percutaneous introduction of the first anchor structure into the coronary sinus of the patient's heart.

25. The apparatus defined in claim 24 further comprising:
means adapted for percutaneous operation of the first anchor structure to secure it to the patient's tissue.

26. The apparatus defined in claim 23 further comprising:
means adapted for percutaneous introduction of the second anchor structure into the patient.

27. The apparatus defined in claim 26 further comprising:
means adapted for percutaneous operation of the second anchor structure to secure it to the patient's tissue.

28. The apparatus defined in claim 23 further comprising:
means for percutaneously operating the linking structure to adjust its length.

29. The apparatus defined in claim 23 wherein the linking structure is selectively engageable to hold a desired length after a length adjustment.

30. The apparatus defined in claim 29 wherein the linking structure is also selectively releasable after engagement to permit further adjustment to a new desired length, which may be greater than the first-mentioned desired length.

31. The apparatus defined in claim 23 wherein the first anchor structure includes a portion that is adapted for enlargement to substantially annularly engage a surrounding tissue annulus.

32. The apparatus defined in claim 23 wherein the second anchor structure includes a portion that is adapted for enlargement to substantially annularly engage a surrounding tissue annulus.

33. The apparatus defined in claim 23 wherein the first anchor structure includes a portion that is adapted to penetrate tissue.

34. The apparatus defined in claim 23 wherein the second anchor structure includes a portion that is adapted to penetrate tissue.

35. The apparatus defined in claim 33 wherein the portion is adapted for threading into tissue.

36. The apparatus defined in claim 34 wherein the portion is adapted for threading into tissue.

37. The apparatus defined in claim 23 wherein the linking structure comprises:
first and second flexible members respectively extending from the first and second anchor structures.

38. The apparatus defined in claim 37 wherein the linking structure further comprises:
an engagement structure adapted to engage the first and second flexible members and to move along at least one of the first and second flexible members.

39. The apparatus defined in claim 38 wherein the engagement structure is adapted to move along the at least one of the flexible members toward the anchor structure from which that flexible member extends and to resist oppositely directed movement along that flexible member.

40. The apparatus defined in claim 39 wherein the engagement structure is adapted for selective operation to not resist the oppositely directed movement.

41. The apparatus defined in claim 23 wherein the linking structure comprises:
first and second complimentary and interengageable ratchet structures on the first and second anchor structures, respectively.

42. The apparatus defined in claim 41 wherein the ratchet structures are configured to allow movement of the first and second support structures toward one another, but to resist oppositely directed movement.

43. The apparatus defined in claim 42 wherein the ratchet structures are adapted for selective operation not to resist the oppositely directed movement.

44. Apparatus for remodeling relatively soft body tissue of a patient comprising:
first and second anchor structures adapted for implanting at respective first and second spaced locations in the body tissue; and
linking structure extending between the first and second anchor structures and having a length between the first and second anchor structures that is adjustable after the first and second anchor structures have been implanted to allow adjustment of spacing between the first and second anchor structures.

45. The apparatus defined in claim 44 wherein at least one of the anchor structures comprises:
a helical structure.

46. The apparatus defined in claim 45 wherein the helical structure has a central longitudinal axis about which the helical structure is adapted for rotation to thread the helical structure into the body tissue.

47. The apparatus defined in claim 46 wherein the helical structure includes at least one barb extending from the helical structure and inclined backwardly relative to a direction in which the helical structure is rotated to thread it into the body tissue, whereby the barb resists unthreading the helical structure from the body tissue.

48. The apparatus defined in claim 44 wherein the body tissue includes a lumen, and wherein at least one of the anchor structures includes a substantially annular structure configured for disposition substantially concentrically in the lumen.

49. The apparatus defined in claim 48 wherein the annular structure includes at least one projection for penetrating a wall of the lumen.

50. The apparatus defined in claim 44 wherein at least one of the anchor structures comprises:
a first portion configured for lying on a surface of the body tissue; and
a second portion inclined relative to the first portion and configured for penetrating the body tissue below the surface.

51. The apparatus defined in claim 50 wherein the at least one anchor structure is the first anchor structure, and wherein the first and second portions form an acute angle whose apex points generally away from the second anchor structure in use.

52. The apparatus defined in claim 50 wherein the second portion includes at least one barb extending from the second portion and inclined backwardly relative to a direction in which the second portion is penetrated into the body tissue, whereby the barb resists withdrawing the second portion from the body tissue.

53. The apparatus defined in claim 50 wherein the body tissue includes a lumen, and wherein the first portion includes a substantially annular structure configured for disposition substantially concentrically in the lumen.

54. The apparatus defined in claim 44 wherein at least one of the anchor structures comprises:
first and second portions that are movable relative to one another so that they can both penetrate the body tissue while the first and second portions are substantially aligned with one another, after which the second portion becomes transverse to the first portion to resist withdrawal of the anchor structure from the body tissue.

55. The apparatus defined in claim 54 wherein the first portion is long enough to permit the second portion to pass completely through the body tissue so that the second portion becomes transverse to the first portion adjacent a surface of the body tissue that is remote from where the anchor structure entered the body tissue.

56. The apparatus defined in claim 45 wherein the at least one anchor structure further comprises:
a further anchor structure that is insertable into the helical structure.

57. The apparatus defined in claim 56 wherein the further anchor structure comprises:
first and second portions that are movable relative to one another so that they can both penetrate the body tissue while the first and second potions are substantially aligned with one another, after which the second portion become transverse to the first portion to resist withdrawal of the further anchor structure from the body tissue.

58. The apparatus defined in claim 57 wherein the at least one of the anchor structures further comprises:
interconnection structure selectively inter-engageable between the helical structure and the further anchor structure to allow the further anchor structure to compress the helical structure in use.

59. The apparatus defined in claim 44 wherein the linking structure is configured to allow shortening of the distance between the first and second anchor structures and to resist reversal of any such shortening.

60. The apparatus defined in claim 59 wherein the linking structure is selectively operable to permit reversal of the shortening.

61. The apparatus defined in claim 44 wherein the linking structure comprises:
a flexible member extending from at least one of the anchor structures.

62. The apparatus defined in claim 44 wherein the linking structure comprises:
first and second flexible members extending from the first and second anchor structures, respectively; and
a cinching structure engageable with the first and second flexible members.

63. The apparatus defined in claim 62 wherein the cinching structure is configured to allow at least one of the flexible members to move through the cinching structure in a first direction but not in an opposite second direction.

64. The apparatus defined in claim 63 further comprising:
instrumentation for severing the first and second members adjacent a side of the cinching structure that is remote from the first and second anchor structures along the first and second flexible members.

65. The apparatus defined in claim 44 wherein the linking structure comprises:
a ratcheting structure.

66. The apparatus defined in claim 65 wherein the ratcheting structure is configured to allow the first and second anchor structures to move toward one another but to resist movement of the first and second structures away from one another.

67. The apparatus defined in claim 66 wherein the ratcheting structure is selectively operable to allow movement of the first and second structures away from one another.

68. The apparatus defined in claim 44 further comprising:
instrumentation for implanting at least one of the anchor structures in the body tissue.

69. The apparatus defined in claim 68 wherein the body tissue is internal to the patient, and wherein the instrumentation is configured for implanting the at least one anchor structure at least partly through a body conduit lumen of the patient.

70. The apparatus defined in claim 68 wherein the body tissue is internal to the patient, and wherein the instrumentation is configured for implanting the at least one anchor structure percutaneously.

71. The apparatus defined in claim 68 wherein the body tissue is internal to the patient, and wherein the instrumentation is configured for implanting the at least one anchor structure at least partly via the circulatory system conduits of the patient.

72. The apparatus defined in claim 44 further comprising:
instrumentation for operating the linking structure.

73. The apparatus defined in claim 72 wherein the body tissue is internal to the patient, and wherein the instrumentation is configured for operating the linking structure at least partly through a body conduit lumen of the patient.

74. The apparatus defined in claim 72 wherein the body tissue is internal to the patient, and wherein the instrumentation is configured for operating the linking structure percutaneously.

75. The apparatus defined in claim 72 wherein the body tissue is internal to the patient, and wherein the instrumentation is configured for operating the linking structure at least partly via the circulatory system conduits of the patient.

76. Apparatus for remodeling the annulus of a patient's mitral valve comprising:
first instrumentation for implanting a first anchor structure in the patient's coronary sinus;
second instrumentation for implanting a second anchor structure in the patient's right atrium; and
third instrumentation for employing linking structure between the first and second anchor structures to shorten the distance between those structures.

77. The apparatus defined in claim 76 wherein at least one of the first, second, and third instrumentations is configured for percutaneous use.

78. The apparatus defined in claim 76 wherein all of the first, second, and third instrumentations are configured for percutaneous use.

79. A method of implanting a structure in body tissue that includes an elongated, laterally curved, body tissue conduit comprising:
providing delivery instrumentation having an elongated portion that is laterally curved to approximately correspond to lateral curvature of the body tissue conduit; and
inserting the delivery instrumentation substantially coaxially into the body tissue conduit so that the lateral curvature of the delivery instrumentation causes the delivery instrumentation to angularly orient itself relative to the body tissue conduit to superimpose the lateral curvature of the delivery instrumentation and the body tissue conduit on one another.

80. The method defined in claim 81 further comprising:
dispensing the structure from the delivery instrumentation with a predetermined angular orientation relative to the lateral curvature of the delivery instrumentation.

81. Apparatus for implanting a structure in a laterally curved, elongated, body tissue conduit comprising:
elongated delivery instrumentation adapted to be received substantially coaxially in the conduit, the delivery instrumentation having lateral curvature corresponding to the lateral curvature of the conduit so that the delivery instrumentation tends to orient itself angularly about its longitudinal axis with its curvature substantially following the curvature of the conduit, the delivery instrumentation being adapted to deliver the structure into the conduit with a predetermined angular orientation about a longitudinal axis of the delivery instrumentation.

82. The apparatus defined in claim 81 wherein the delivery instrumentation is laterally flexible.

83. The apparatus defined in claim 81 wherein the lateral curvature is in a relatively distal portion of the delivery instrumentation, and wherein a more proximal portion of the delivery instrumentation has additional lateral curvature for facilitating entry of the distal portion into the body tissue conduit.

84. The apparatus defined in claim 83 wherein the lateral curvature is compound with the additional lateral curvature.

85. Apparatus for use with a laterally curved, elongated body tissue conduit comprising:
elongated instrumentation adapted to be received substantially coaxially in the conduit, the instrumentation having lateral curvature corresponding to the lateral curvature of the conduit so that the instrumentation tends to orient itself angularly about its longitudinal axis with its curvature substantially following the curvature of the conduit.

86. The apparatus defined in claim 85 wherein the instrumentation includes means for delivering an implant with a predetermined angular relationship to the lateral curvature of the instrumentation.

87. The apparatus defined in claim 86 wherein the means for delivering delivers the implant into the conduit.

88. The apparatus defined in claim 86 wherein the means for delivering delivers the implant at a location outside the conduit.

89. Apparatus for remodeling a patient's left ventricle comprising:
first instrumentation for implanting a first anchor structure at a first location in the patient's left ventricle;
second instrumentation for implanting a second anchor structure at a second location in the patient's left ventricle spaced from the first location; and
third instrumentation for employing linking structure between the first and second anchor structures to decrease spacing between the first and second anchor structures.

90. The apparatus defined in claim 89 wherein at least one of the first, second, and third instrumentations is configured for percutaneous use.

91. The apparatus defined in claim 89 wherein all of the first, second, and third instrumentations are configured for percutaneous use.

92. A method of remodeling relatively soft body tissue of a patient comprising:
implanting first and second anchor structures at respective first and second spaced locations in the body tissue; and
using a linking structure between the first and second anchor structures to change the spacing between the first and second anchor structures.

93. The method defined in claim 92 wherein the body tissue is the mitral valve annulus of the patient, and wherein at least one of the anchor structures is implanted via the coronary sinus of the patient.

94. The method defined in claim 93 wherein one of the anchor structures is implanted via the coronary sinus, and the other of the anchor structures is implanted in the patient's right atrium.

95. The method defined in claim 92 wherein the body tissue is the left ventricle of the patient, and wherein the first and second anchor structures are implanted in the left ventricle.
